(19) United States
(12) Patent Application Publication
(10) Pub. No.: US 2012/0128711 A1
(43) Pub. Date: May 24, 2012

(54) ANTI-INFLAMMATORY APPROACH TO PREVENTION AND SUPPRESSION OF POST-TRAUMATIC STRESS DISORDER, TRAUMATIC BRAIN INJURY, DEPRESSION AND ASSOCIATED DISEASE STATES

(75) Inventors: Marvin S. Hausman, Stevenson, WA (US); Malli Rao Varanasi, San Clemente, CA (US)

(73) Assignee: TOTAL NUTRACEUTICAL SOLUTIONS, INC., Stevenson, WA (US)

(21) Appl. No.: 13/363,579

(22) Filed: Feb. 1, 2012

Related U.S. Application Data
(63) Continuation-in-part of application No. 12/887,276, filed on Sep. 21, 2010.
(60) Provisional application No. 61/438,483, filed on Feb. 1, 2011.
(60) Provisional application No. 61/277,150, filed on Sep. 21, 2009.
(60) Provisional application No. 61/280,578, filed on Nov. 5, 2009.
(60) Provisional application No. 61/335,394, filed on Jan. 6, 2010.

(51) Int. Cl. A61K 36/06; A61P 29/00; A61P 25/00; A61K 36/9066

(52) U.S. Cl. 424/195.15

(57) ABSTRACT

A composition and methods of use are disclosed for prevention and control of inflammation and oxidative stress and various associated medical conditions, including PTSD, chronic depression and traumatic brain injury. A composition providing a phytonutrient and enriched mushrooms having enhanced Vitamin D and ergothioneine are disclosed. The combined use of an enriched mushroom and phytonutrient provide a synergistic effect on cellular longevity and/or cellular rejuvenation of subjects with both a normal and nutritionally deficient diets, improved tolerance to oxidative and/or inflammatory stress, and increased cellular longevity and/or cellular rejuvenation as a result of the neutralization of free radicals and prevention of chronic inflammation.

### CROSS-REFERENCE TO RELATED APPLICATIONS

This application claims priority under 35 U.S.C. §119 of a provisional application Ser. No. 61/438,483 filed Feb. 1, 2011, which application is hereby incorporated by reference in its entirety.

This application is also a continuation-in-part application of Ser. No. 12/887,276 filed Sep. 21, 2010, titled Vitamin D2 Enriched Mushrooms and Fungi for Treatment of Oxidative Stress, Alzheimer's Disease and Associated Disease States, which claims priority under 35 U.S.C. §119 of provisional application Ser. Nos. 61/277,150 filed Sep. 21, 2009, 61/280,578 filed Nov. 5, 2009 and 61/335,394 filed Jan. 6, 2010, herein incorporated by reference in their entirety.

### FIELD OF THE INVENTION

This invention relates to a nutritional product for use as a dietary supplement or other food product for preventing, suppressing or treating post-traumatic stress disorder (PTSD), traumatic brain injury, depression and associated neuroinflammatory and oxidative stress conditions, by the use of phytonutrients and enriched mushroom antioxidants and bionutrients to neutralize free radicals and prevent chronic inflammation. The invention further relates to enhancing cellular longevity and/or cellular rejuvenation through control of oxidative stress and other inflammatory conditions.

### BACKGROUND OF THE INVENTION

Neuroinflammatory conditions are interactive processes involving inflammation, free radicals, reactive oxygen species (ROS) and oxidative stress. Free radicals (or ROS) are chemical species (such as an atom or molecule) with one or more unpaired electrons in its valance shell, making then unstable, short lived and highly reactive. These species react quickly with other compounds to obtain an additional electron to gain stability. Free radicals exist in various chemical forms and many are also used as biologic markers of inflammatory conditions, including for example, cytokines such as IL-2, TNF-alpha, nitric oxide, hydrogen peroxide and heat shock protein. It is known that free radicals cause tissue damage and that endogenous free radicals are neutralized by endogenous antioxidants. In inflammatory diseases, such as PTSD, acute and chronic depression and traumatic brain injury, free radicals perpetuate tissue damage.

According to the DSM-IV-TR, post-traumatic stress disorder, often abbreviated as PTSD, is a complex disorder in which the affected person's memory, emotional responses, intellectual processes, and nervous system have all been disrupted by one or more traumatic experiences. Often the disorder is understood to manifest as a normal reaction to abnormal events. Persons with PTSD have reported traumatic experiences, such as witnessing someone being badly hurt or killed; involvement in a severe natural disaster or life-threatening accident; and military combat. PTSD has become more widespread in the general population and according to the National Comorbidity Survey conducted between 1990 and 1992, it is estimated that the lifetime prevalence among adult Americans is 7.8%, with women (10.4%) twice as likely as men (5%) to be diagnosed with PTSD at some point in their lives.

There are also certain populations at greater risk of developing PTSD. For example, the lifetime prevalence of PTSD among persons living in depressed urban areas or on Native American reservations is estimated at 23%. For victims of violent crimes, the estimated rate is 58%. In addition, significant research is ongoing to understand the association of PTSD and military veterans. It is estimated that the lifetime prevalence of PTSD among Vietnam veterans is 30.9% for men and 26.9% for women. An additional 22.5% of the men and 21.2% of the women Vietnam veterans have been diagnosed with partial PTSD at some point in their lives. The lifetime prevalence of PTSD among veterans of World War II and the Korean War is estimated at 20%.

It is suggested that PTSD is linked to a variety of additional healthcare conditions, including for example, heart disease, chronic pain, fatigue, metabolic/bowel disorders, and dementia. Several recent scientific articles have suggested that there are associated endocrine and immune function changes in people with PTSD, and especially those with associated chronic depression. A common causative link to many if not all of the physiologic disorders associated with PTSD is inflammation, a response of body tissues to injury or irritation that is characterized by pain, swelling, redness, and heat. The body produces inflammatory free radicals triggering oxidative stress and inflammation.

Depression is also believed to be linked to chronic brain inflammation. (Karen Wager-Smith, Athina Markou. Depression: A repair response to stress-induced neuronal micro damage that can grade into a chronic neuroinflammatory condition? Neuroscience & Biobehavioral Reviews, 2010; DOI: 10.1016/j.neubiorev.2010.09.010). There remains a need for treatments for both chronic and acute depression that is linked to brain inflammation.

Traumatic brain injury (TBI) is an additional type of brain inflammation with primary body injury relating to the head and associated with concussions or internal organ injury. TBI is frequently caused by a sport- or recreation-related injury; an estimated 1.1 million persons a year, based on CDC and U.S. hospital emergency department statistics, seek hospital treatment for TBI. (CDC: MMWR Weekly. Nonfatal Traumatic Brain Injuries from Sports and Recreation Activities, Jul. 27, 2007; 56(29); 733-737). The highest rates of sports- and recreation-related TBI injuries are associated with males and females between the ages of 10-14 years. Acute brain inflammation is most often implicated with a TBI, and the majority of TBIs are categorized as mild. However, even mild TBI can affect a person's ability to return to school or work and can result in long-term cognitive or other problems. In addition, repeated and/or severe TBIs can result in physical, cognitive, behavioral, or emotional problems and lead to various long-term, negative health effects, such as memory loss, behavioral changes and increased risk for depression. As a result, prevention measures are desirable for TBIs. In addition to enhanced safety measures (such as protective equipment, including helmets) to prevent TBIs, additional prevention and treatment measures are needed.

Turmeric (Curcuma longa) is a phytonutrient (also commonly referred to as a phytochemical) originating from the rhizomatous herbaceous perennial plant of the ginger family, Zingiberaceae. It is native to tropical South Asia and needs temperatures between 20° C. and 30° C. and a considerable amount of annual rainfall to thrive. Although most usage of turmeric is in the form of root powder, in some regions, leaves of turmeric are used to wrap and cook food. This usually takes place in areas where turmeric is grown locally, since the leaves used are freshly picked. When not used fresh, the rhizomes are boiled for several hours and then dried in hot ovens, after which they are ground into a deep orange-yellow powder commonly used as a spice in curries and other South Asian and Middle Eastern cuisine, for dyeing, and to impart color to mustard condiments.

Its active ingredient is curcumin and it has a distinctly earthy, slightly bitter, slightly hot peppery flavor and a mustardy smell. Turmeric (coded as E100 when used as a food additive) is used to protect food products from sunlight. The oleoresin is used for oil-containing products. The curcumin/polysorbate solution or curcumin powder dissolved in alcohol is used for water-containing products. Over-coloring, such as in pickles, relishes, and mustard, is sometimes used to compensate for fading. It is also commonly used in canned beverages and baked products, dairy products, ice cream, yogurt, yellow cakes, orange juice, biscuits, popcorn color, sweets, cake icings, cereals, sauces, gelatins, etc.

The medicinal properties of turmeric have been recognized in some South Asian countries, including use as a readily available antiseptic and antibacterial for cuts, burns and bruises. It is taken in some Asian countries as a dietary supplement, which allegedly helps with stomach problems and other ailments. It is popular as a tea in Okinawa, Japan. In India, it is also used as an anti-inflammatory agent, and remedy for gastrointestinal discomfort associated with irritable bowel syndrome, and other digestive disorders. In Afghanistan and northwest Pakistan, turmeric is applied to a piece of burnt cloth, and placed over a wound to cleanse and stimulate recovery. Turmeric paste is traditionally used by Indian women to remove superfluous hair and improve the skin and act as an anti-aging agent. In addition, turmeric is believed to improve the skin tone and tan and is currently used in the formulation of some sunscreens.

Turmeric is currently being investigated for possible benefits in Alzheimer's disease, cancer, arthritis, and other clinical disorders. Research activity into curcumin and turmeric is increasing which is consistent with the increase in sales of supplements of 35% from 2004. In addition, the U.S. National Institutes of Health currently has registered 19 clinical trials to study use of dietary turmeric and curcumin for a variety of clinical disorders.

The valuable health benefits of mushrooms are disclosed in U.S. patent application Ser. No. 12/887,276, titled Vitamin D2 Enriched Mushrooms and Fungi for Treatment of Oxidative Stress, Alzheimer's disease and Associated Disease States, which is herein incorporated by reference in its entirety. Mushrooms are a valuable health food—low in calories, high in vegetable proteins, chitin, iron, zinc, fiber, essential amino acids, vitamins and minerals. They are also an excellent source of organic selenium compounds, riboflavin, pantothenic acid, copper, niacin, potassium and phosphorous. Selenium is needed for the proper function of the antioxidant system, which works to reduce the levels of damaging free radicals in the body. Selenium is a necessary cofactor of one of the body's most important internally produced antioxidants, glutathione peroxidase, and also works with vitamin E in numerous vital antioxidant systems throughout the body. Mushrooms are also a primary source of natural Vitamin D, in the form of D2, which is naturally present in very few foods. Most other natural food sources of Vitamin D, in the form Vitamin D3, are of animal, poultry or seafood origin.

Vitamin D is a fat-soluble vitamin that is naturally present in very few foods, added to others, and available as a dietary supplement. Vitamin D comes in two forms (D2 and D3) which differ chemically in their side chains. These structural differences alter their binding to the carrier protein Vitamin D binding protein (DBP) and their metabolism, but in general the biologic activity of their active metabolites is comparable. It is also produced endogenously when ultraviolet rays from sunlight strike the skin and trigger Vitamin D synthesis. So one must either ingest Vitamin D or sit in the sun and soak up UV rays, so that it may be synthesized endogenously. The risks of sun exposure have gained much attention lately, and the association of sun exposure with pre-cancerous (actinic keratosis) and cancerous (basal cell carcinoma, squamous cell carcinoma and melanoma) skin lesions—caused by loss of the skin's immune function, fine and coarse wrinkling of the skin, freckles, discoloration of the skin, and Elastosis—the destruction of the elastic tissue causing lines and wrinkles is well documented. Thus as people become more sensitive to the dangers of UV exposure, other dietary sources of Vitamin D become increasingly important for maintaining health.

There are two basic types of Vitamin D. Ergosterol is the basic building block of Vitamin D in plants and fungi. Cholesterol is the basic building block of Vitamin D in humans. When ultraviolet light from the sun hits the leaf of a plant or fungal tissue, ergosterol is converted into ergocalciferol, or Vitamin D2. In just the same way, when ultraviolet light hits the cells of our skin, one form of cholesterol found in our skin cells-called 7-dehydrocholesterol-can be converted into cholecalciferol, a form of Vitamin D3. The liver and other tissues metabolize Vitamin D, whether from the skin or oral ingestion, to 25OHD, the principal circulating form of Vitamin D, by the enzyme CYP27B1, the 25OHD-lahydroxylase. 25OHD is then further metabolized to 1,25(OH)₂D principally in the kidney, although other tissues such as epidermal keratinocytes and macrophages contain this enzymatic activity. 1,25(OH)₂D is the principal hormonal form of Vitamin D, responsible for most of its biologic actions.

Vitamin D has many roles in human health, including modulation of neuromuscular and immune function and reduction of inflammation. Many genes encoding proteins that regulate cell proliferation, differentiation, and apoptosis are modulated in part by Vitamin

D. Many laboratory-cultured human cells have Vitamin D receptors and some convert 25(OH)D to 1,25(OH)₂D. It remains to be determined what cells, tissues, and organs in the human body contain either D2, D3, or both vitamin receptors and what additional cells with Vitamin D receptors in the intact human can carry out this conversion from 25(OH)D to 1,25(OH)₂D.

It is an object of the present invention to provide a dietary supplement or other food or beverage products which are high in nutritional values, particularly Vitamin D2, ergothioneine, and optionally turmeric and/or Omega-3.

It is another object of the invention to provide dietary supplements or other food or beverage products for use in prevention, suppression or treatment of post-traumatic stress disorder and associated neuroinflammatory conditions involving chronic inflammation.

It is a still further object of the invention to provide novel uses for anti-inflammatory agents for prevention, treatment and suppression of PTSD, chronic depression, traumatic brain injury and associated neuroinflammatory conditions.

These and other objects of the present invention will become apparent from the description of the invention which follows.

### SUMMARY OF THE INVENTION

Prevention, treatment and suppression of neuroinflammatory and oxidative stress associated conditions are provided according to the invention as a result of a clear understanding of the interactive processes involving inflammation, free radicals, reactive oxygen species (ROS) and oxidative stress.

This invention creates an improved food or supplement product with a naturally enriched Vitamin D, ergothioneine and turmeric (or other phytonutrient) nutritional profile. The product combines a phytonutrient, such as turmeric or omega-3 with a Vitamin D enriched mushroom substrate, namely a mushroom or other fungi having enhanced content of Vitamin D or its analogs or derivatives. Applicants have discovered the dosage and timing of supplementation required to provide the highest benefit of increased turmeric and Vitamin D content, for the prevention, suppression and/or treatment of neuroinflammatory conditions, such as post-traumatic stress disorder, traumatic brain injury and depression.

In an embodiment, the Vitamin D, ergothioneine and turmeric (or other phytonutrient antioxidant) supplement or food product is used in animal feed or as a nutritional supplement in humans. The enriched supplement or food product prevents, reduces and/or suppresses neuroinflammatory and oxidative stress associated conditions, such as post-traumatic stress disorder (PTSD), traumatic brain injury and depression. The lethality of the conditions are reduced as a result of the role of the anti-inflammatory effects of turmeric and the role of Vitamin D2 in enhancing cellular longevity and/or cellular rejuvenation under stressful conditions involving oxidative stress conditions and the generation of free radicals. Overall, the uses according to the invention improve and increase survival in biologic models and organisms with neuroinflammatory conditions. The reported results are observed, surprisingly, in contrast to either turmeric or Vitamin D2 or D3 fed alone, despite some benefit observed from the use of turmeric alone.

In a further embodiment, the invention includes pharmaceutical compositions for prevention of, treatment for, and resistance to the effects of neuroinflammation and oxidative stress, and disease states such as PTSD, traumatic brain injury, depression and other associated conditions.

### DETAILED DESCRIPTION OF THE FIGURES

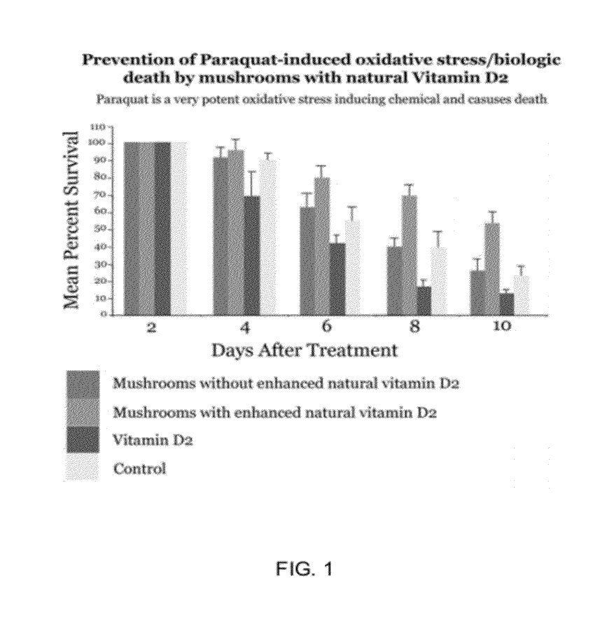

FIG. 1 shows the prevention of Paraquat-induced oxidative stress/biologic death by fungi with naturally-enriched Vitamin D2 based on mean percent survival.

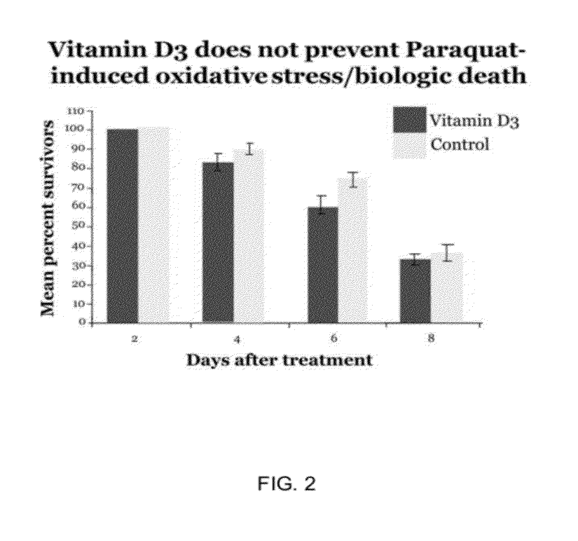

FIG. 2 shows the unexpected result that although mushroom contained Vitamin D2 is able to counteract and/or neutralize the oxidative stress effect and resulted in a 30% increase in survival, pure Vitamin D2 and Vitamin D3 by itself have no effect on survival.

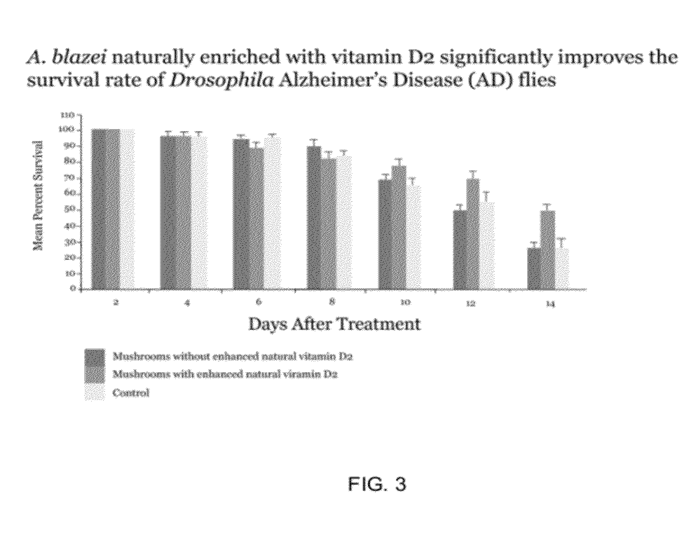

FIG. 3 shows the improvement in survival of Alzheimer's disease (AD) flies given A. blazei enriched with Vitamin D2, having a survival rate nearly double that of the control or A. blazei without any enrichment.

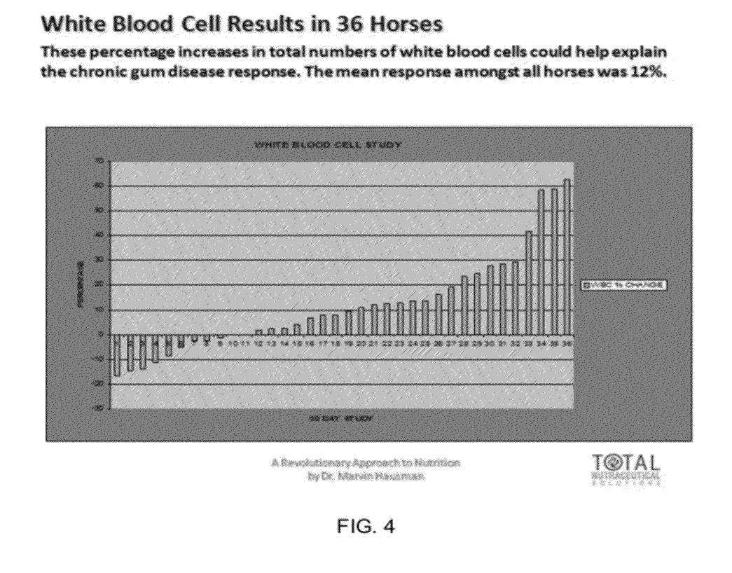

FIG. 4 shows results of decreased severity of gum disease in horses treated with mushrooms containing Ergothioneine and Vitamin D and the clinical marker of increased number of WBC.

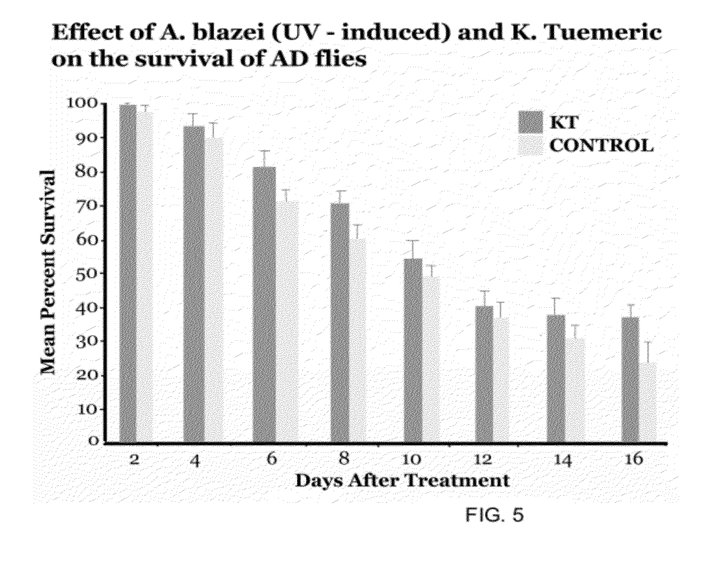

FIG. 5 shows AD Drosophila survival with and without turmeric. The results show that turmeric significantly enhanced survival.

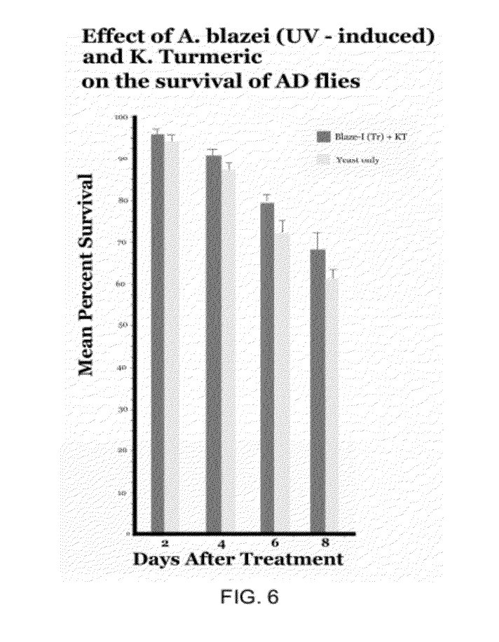

FIG. 6 is a graph showing AD Drosophila survival with and without the turmeric and Vitamin D2-enriched mushroom. The results show that the combination significantly enhances survival.

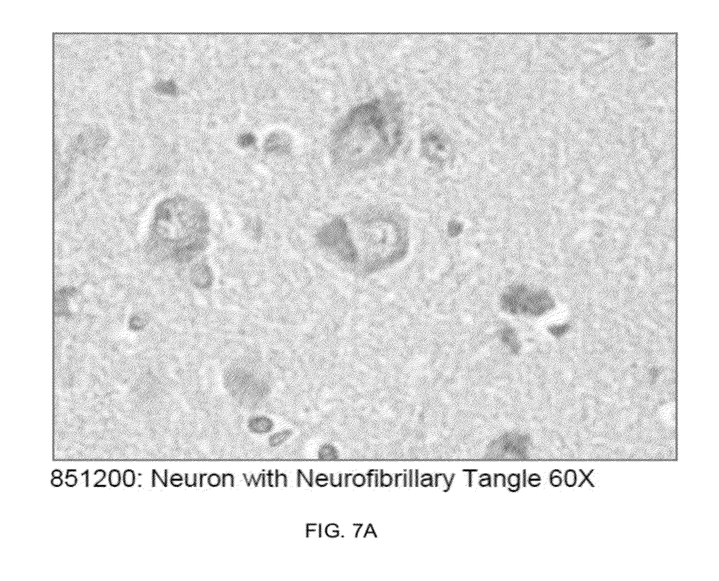

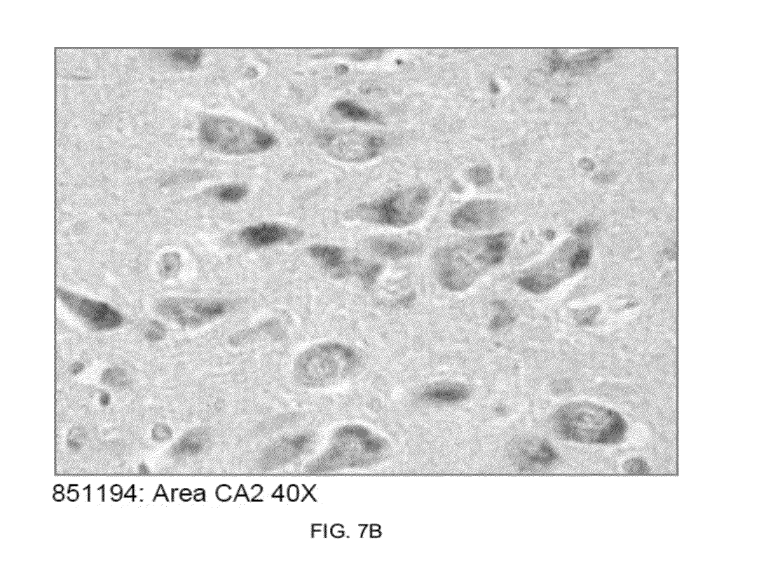

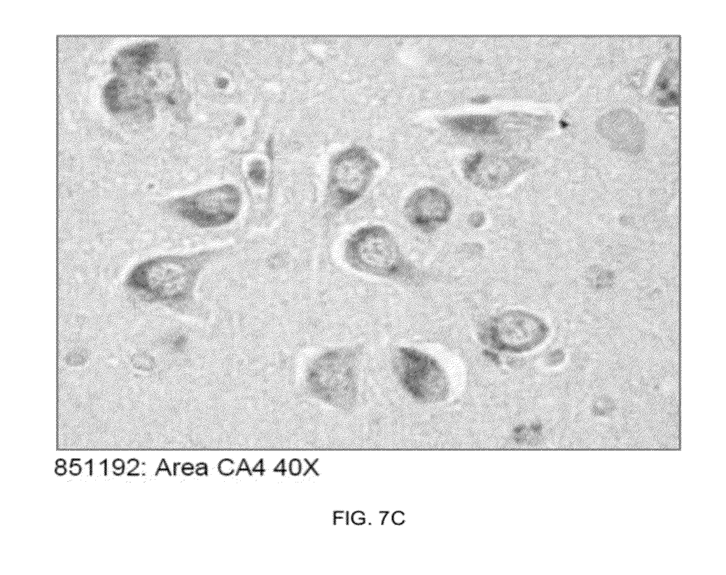

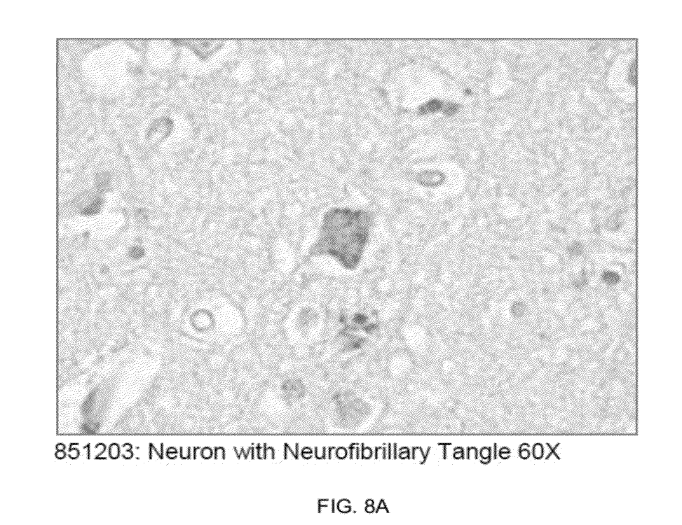

FIGS. 7A-C show immunohistochemistry slides showing the presence/levels of the ergothioneine transporter (SLC22A4) in brain tissue samples of patients with Alzheimer's disease.

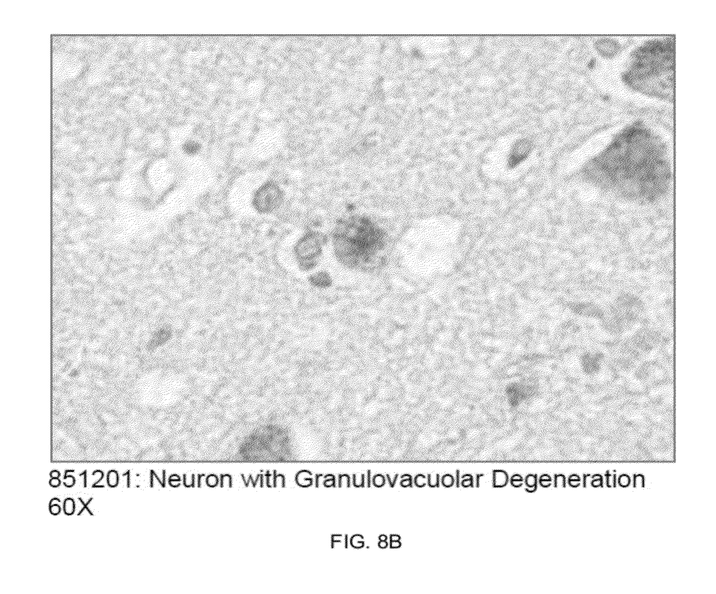

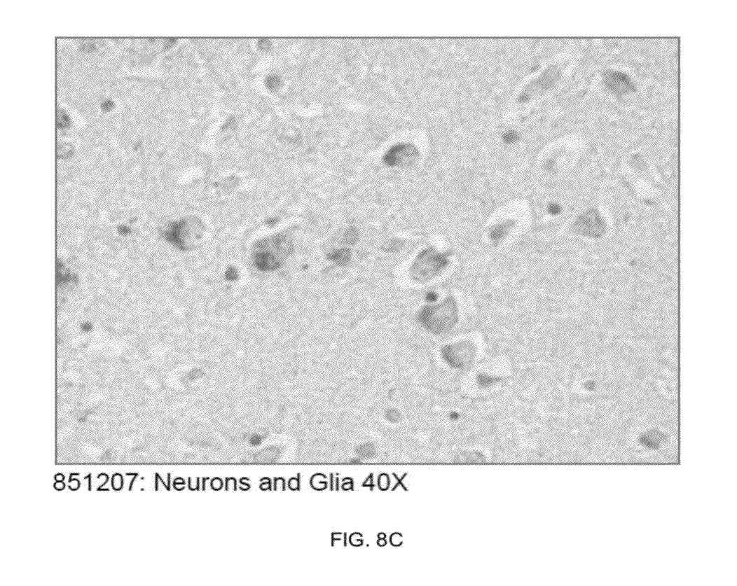

FIGS. 8A-C show additional immunohistochemistry slides showing the presence/levels of the ergothioneine transporter (SLC22A4) in brain tissue samples of patients with Alzheimer's disease.

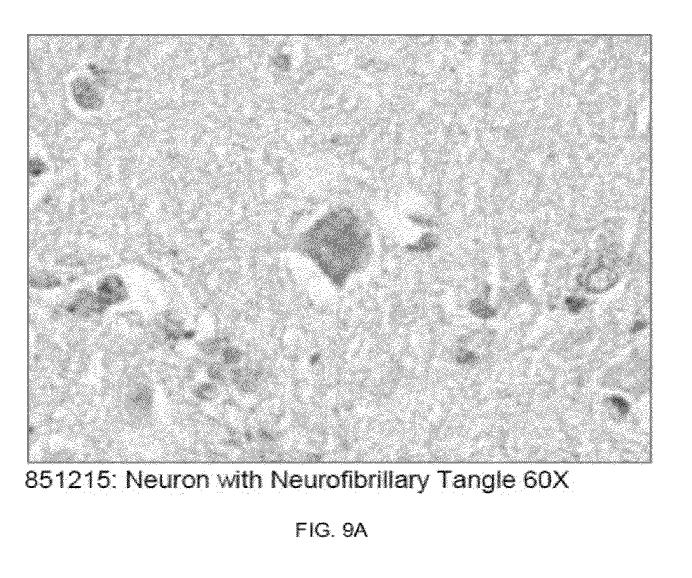

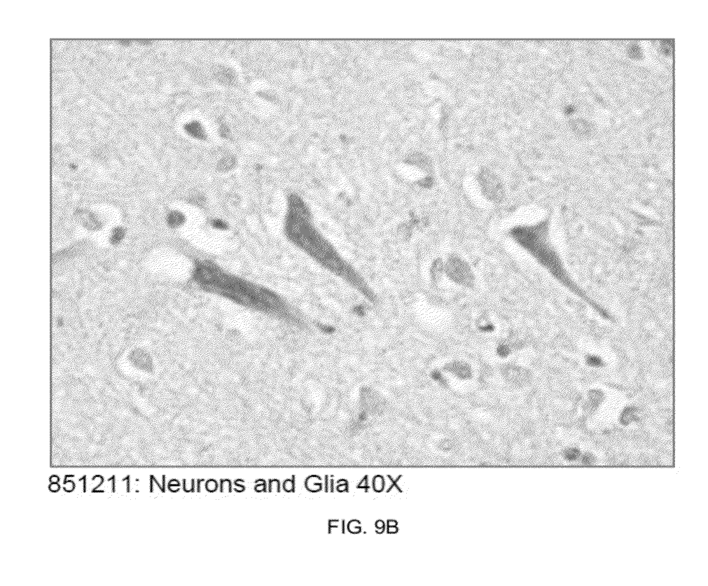

FIGS. 9A-B show additional immunohistochemistry slides showing the presence/levels of the ergothioneine transporter (SLC22A4) in brain tissue samples of patients with Alzheimer's disease.

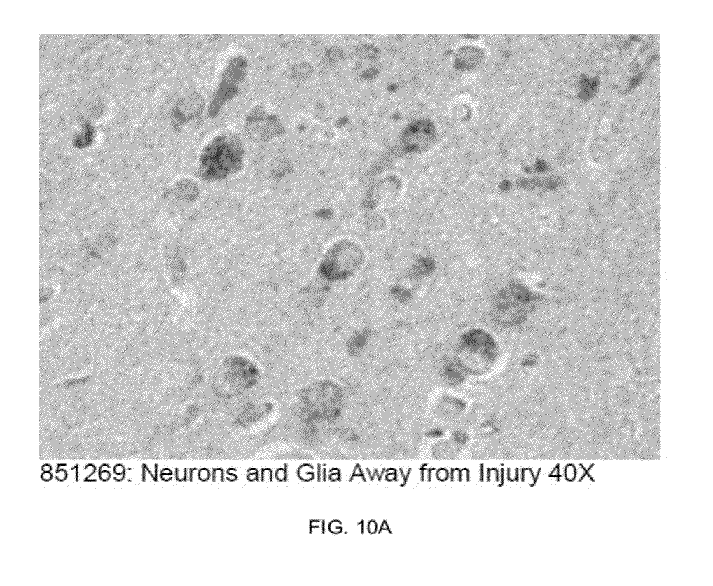

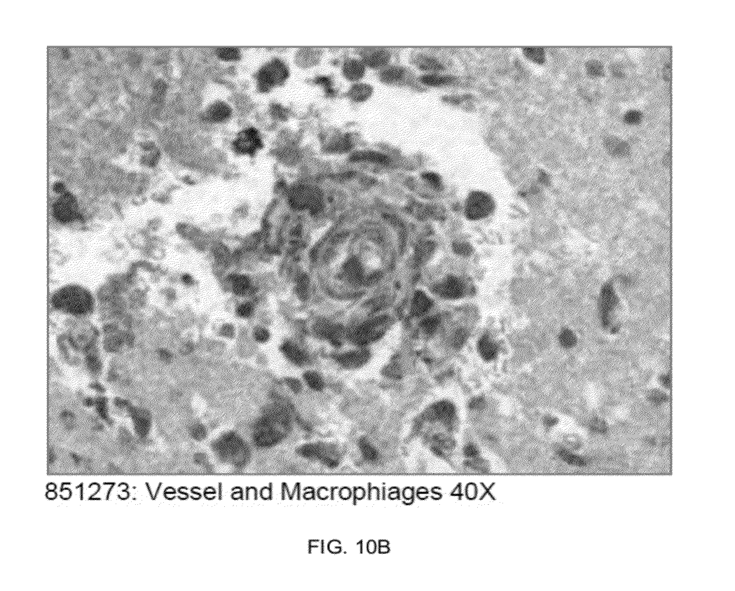

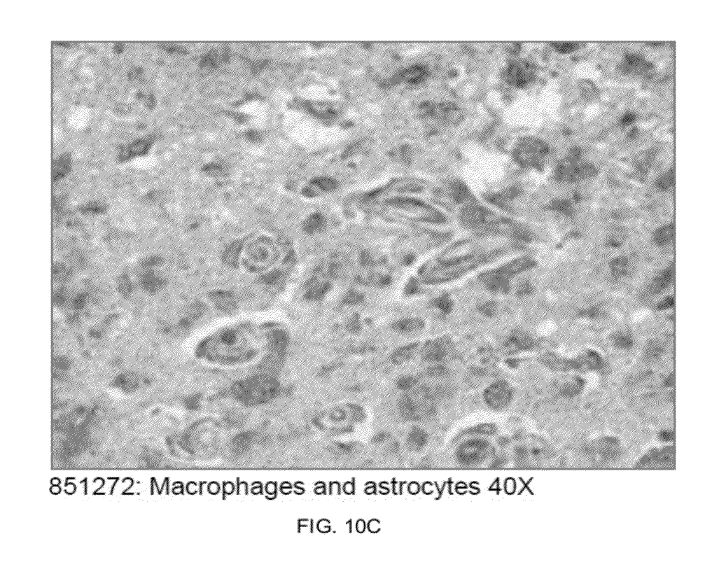

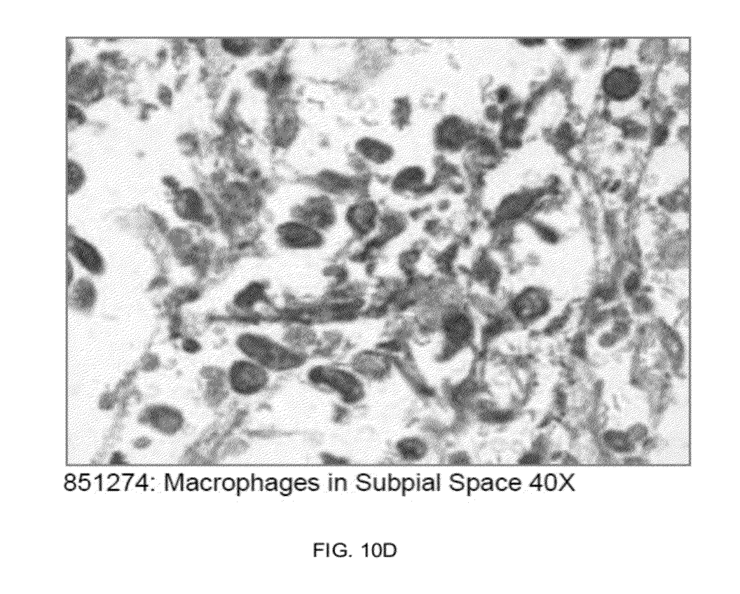

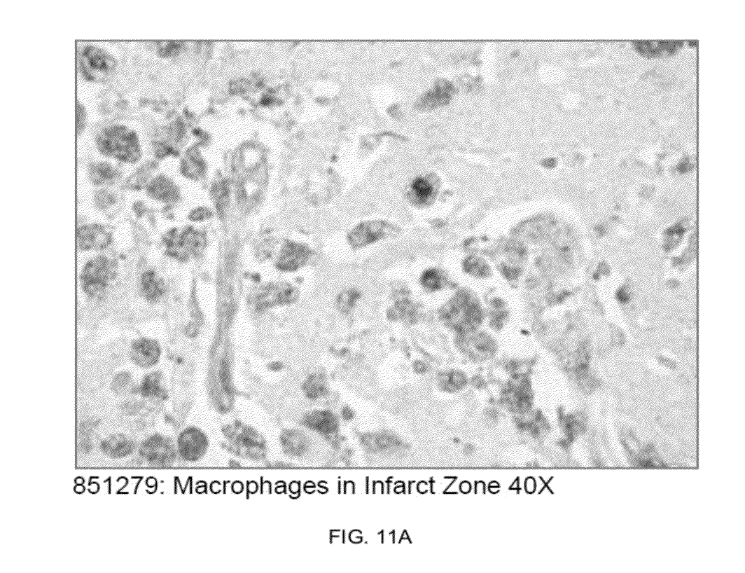

FIGS. 10A-D show immunohistochemistry slides showing the presence/levels of the ergothioneine transporter (SLC22A4) in brain tissue samples of stroke patients.

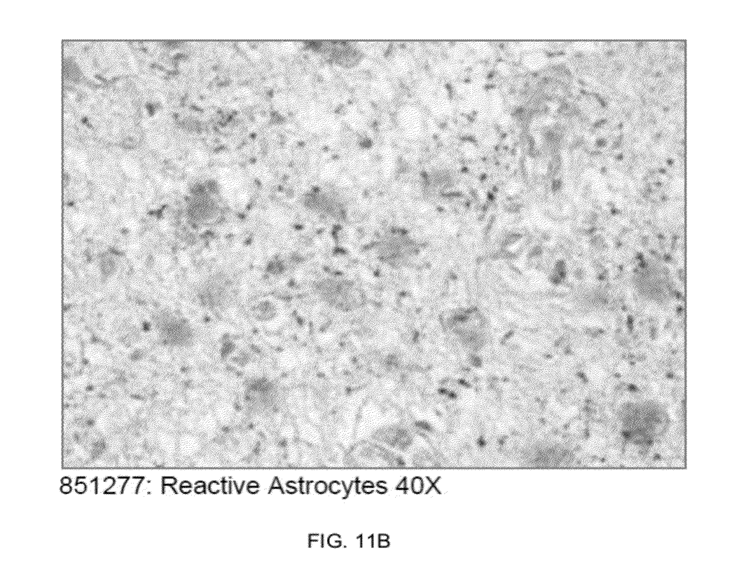

FIGS. 11A-B show additional immunohistochemistry slides showing the presence/levels of the ergothioneine transporter (SLC22A4) in brain tissue samples of stroke patients.

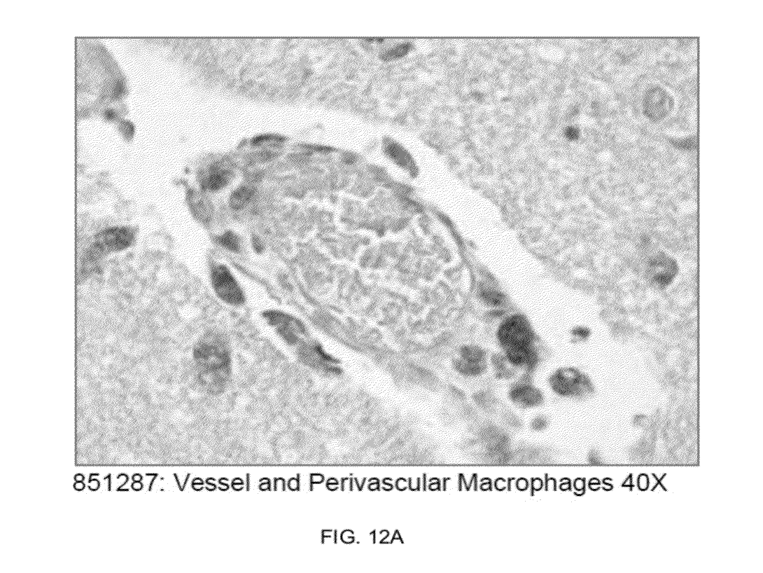

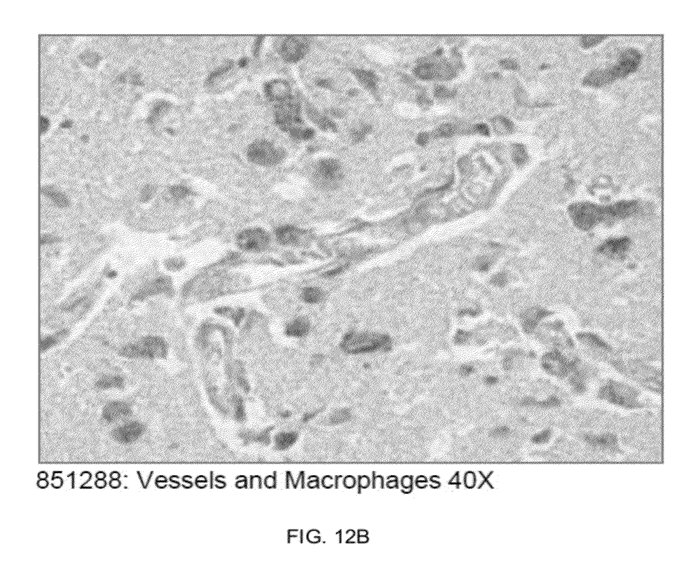

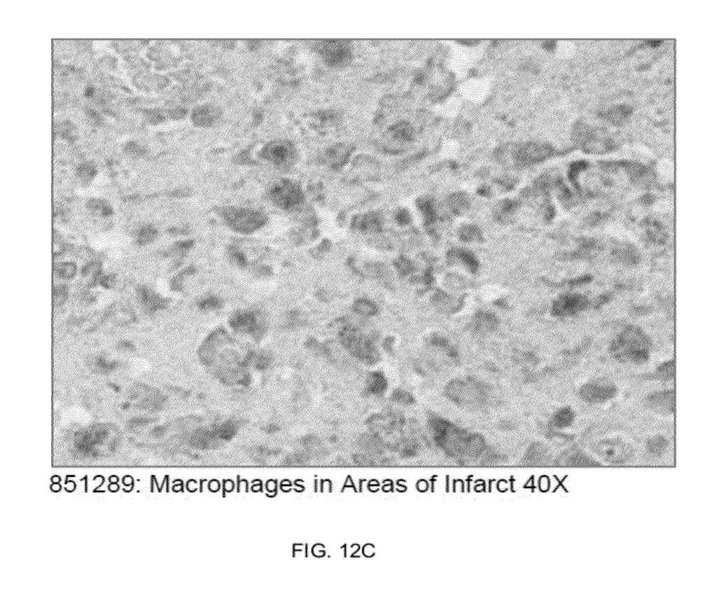

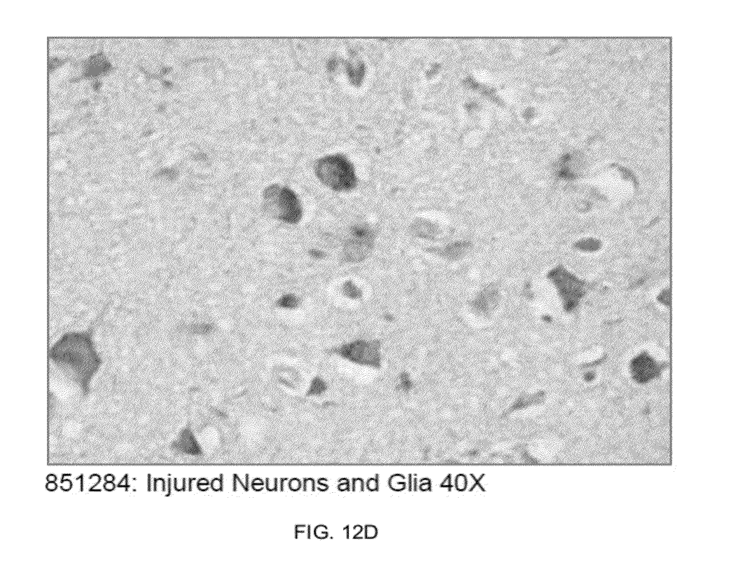

FIGS. 12A-D show further immunohistochemistry slides showing the presence/levels of the ergothioneine transporter (SLC22A4) in brain tissue samples of stroke patients.

### DETAILED DESCRIPTION OF THE INVENTION

The embodiments of this invention are not limited to particular embodiments for compositions and use of phytonutrients and enriched mushrooms for neuroinflammatory conditions involving chronic inflammation and other oxidative stress related conditions, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form. Numeric ranges recited within the specification are inclusive of the numbers defining the range and include each integer within the defined range.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities refers to variation in the numerical quantity that can occur.

As used herein the term “mushroom” or “filamentous fungi” shall be interpreted to include all tissues, cells, organs of the same, including but not limited to mycelium, spores, gills, fruiting body, stipe, pileus, lamellae, basidiospores, basidia, and the like.

As used herein the term “naturally-enhanced” with respect to mushrooms and Vitamin D, shall mean pulsed UV irradiated mushrooms produced by the methods disclosed herein.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The claimed invention improves upon prior research using Vitamin D-enhanced supplements for treating conditions associated with oxidative stress. Previous research showed that exposing mushrooms to constant ultraviolet light can produce Vitamin D2 by converting the naturally-occurring ergosterol to Vitamin D2. However, there were significant disadvantages associated with the deleterious effects on appearance, tissue browning, increased length of exposure time required by conventional sources of UV light and associated regulatory and commercial processing concerns. Prior research further showed pulsed UV-light treatments at very high levels for long period of time (30 seconds or more) reduced bacterial populations in fresh mushrooms, suggesting that Vitamin D2 content in mushrooms could be rapidly increased using pulsed UV-light. Improvements to the pulsed IV-light treatments of mushrooms are combined with phytonutrient compositions for the unexpected prevention, treatment and suppression of neuroinflammatory conditions.

Compositions

According to an embodiment of the invention, a pharmaceutical composition for treating a disease state associated with neuroinflammation and/or oxidative stress comprises ergothioneine and a UV irradiated, Agaricus fungi, tissue, substrate or component thereof with higher levels of Vitamin D2 than a non-irradiated product and a pharmaceutically-acceptable carrier. According to a further embodiment of the invention, the pharmaceutical composition may further comprise turmeric, omega-3 or an alternative antioxidant. Additional antioxidants may either replace or supplement the turmeric and ergothioneine in the compositions according to the invention. According to a preferred embodiment, the compositions of the invention comprise a phytonutrient antioxidant in addition to the fungi component to provide a combined synergistic response.

Turmeric is available in various forms contains up to 5% essential oils and up to 5% curcumin, a polyphenol. Curcumin is the active substance of turmeric and curcumin is known as C.I. 75300, or Natural Yellow 3. The systematic chemical name is (1E,6E)-1,7-bis(4-hydroxy-3-methoxyphenyl)-1,6-heptadiene-3,5-dione and exists in tautomeric forms—keto and enol—shown below. It can exist at least in two tautomeric forms, keto and enol. The keto form is preferred in solid phase, whereas the enol form is preferred in solution. Curcumin is a pH indicator, turning yellow in acidic solutions (pH<7.4) and turning bright red in basic (pH>8.6) solutions.

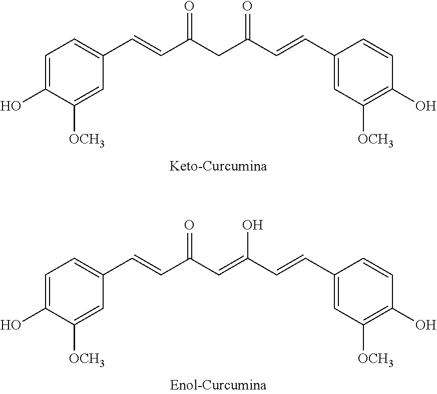

Ergothioneine is a naturally-occurring amino acid. The compound is a thiourea derivative of histidine. The compound is available from Actinobacteria and filamentous fungi. Ergothioneine is a natural antioxidant but is unable to be made in human cells, rather it is absorbed from the diet. Ergothioneine for use in compositions according to the invention may be obtained from an independent bionutrient source, or be obtained from the Vitamin D enriched mushrooms disclosed herein.

Vitamin D enriched mushrooms according to the invention are pulsed with UV light at lower ranges and for very brief periods have increases by as much as 800 times the % DV (percent daily value) of Vitamin D content, per serving with no deleterious effects on the morphology or appearance of the mushroom. Pulsed UV-light treatments to increase Vitamin D₂ content in mushrooms were conducted with a laboratory scale, pulsed light sterilization system (SteriPulse®-XL 3000, Xenon Corporation, Woburn, Mass.) that is present in the Department of Agricultural Biological Engineering at Penn State. While applicants postulate that it is the UVB component of the Xenon pulsed light system that is responsible for the effects of the invention, it should be noted that the system uses pulsed light which includes the entire spectrum of light and may also include other components that contribute to the effects demonstrated herein and which are intended to be within the scope of the invention.

The Vitamin D enriched mushrooms according to the invention contain high levels the bioactive nutrients in addition to Vitamin D. For example, according to an embodiment the mushrooms may also contain high levels of L-Ergothioneine and beta-glucans.

Any type of mushroom, mushroom part, component, fungi or even used substrate for cultivating mushrooms, with ergosterol present may be used. This includes all filamentous fungi where ergosterol has been shown to be present and includes the use of tissues such as the mycelia, spores or vegetative cells. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, Phytophthora achlya, Flammulina, Melanoleuca, Agrocybe, Morchella, Mastigomycotina, Auricularia, Gymnopilus, Mycena, Boletus, Gyromitra, Calvatia, Kuegneromyces, Phylacteria, Cantharellus, Lactarius, Clitocybe, Lentinula (Lentinus), Lepiota, Tuber, Tremella, Drosophia, Leucocoprinus, Tricholoma, Dryphila, Marasmius, and Volvariella.

Non-limiting examples of other fungal genera, including fermentable fungi, include: Alternaria, Endothia, Neurospora, Aspergillus, Fusarium, Penicillium, Blakeslea, Monascus, Rhizopus, Cephalosporium, Mucor, and Trichoderma

In addition, the solid substrate can be any part of the mushroom or mold, including the mycelia, spores etc., so long as ergosterol is present in at least part of the tissue or cells. In yet another embodiment, the spent mushroom substrate upon which mushrooms are cultivated, was enriched in Vitamin D using pulsed UV light according to the invention. As one skilled in the art shall ascertain, mushrooms are usually produced by first preparing a substrate, such as corn, oats, rice, millet or rye or various combinations, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product. According to the invention, this spent substrate may also be enriched in Vitamin D upon application of pulsed UV irradiation.

Food Compositions

An embodiment of the present invention also provides medical foods comprising ergothioneine, and the enriched mushrooms of the invention including extracts, fractions thereof or compounds thereof or any combination thereof. The foods and other compositions of the invention may optionally exclude turmeric, omega-3 or an alternative antioxidant. The food compositions according to the invention may comprise enriched mushrooms from a variety of fungi sources as disclosed according to embodiments herein this description.

The medical food is compounded for the amelioration of a disease, disorder or condition associated with or caused by neuroinflammation, chronic inflammation and/or oxidative stress. According to a preferred embodiment of the invention, food compositions are intended for human consumption as supplementation for their anti-inflammation and anti-oxidant effects. Ranges of the amounts of each component of the food compositions can be adjusted as necessary for the supplementation of individual patients and according to the specific condition treated. Dose ranges can be adjusted as necessary for the treatment of individual patients and according to the specific condition treated, an example of personalized medicine. Any variations in the amount of turmeric, and/or enriched mushrooms may be utilized according to the desired composition formulation.

The food composition according to the invention may be prepared by any of the well-known techniques known by those skilled in the art, consisting essentially of admixing the components, optionally including one or more accessory ingredients. In one embodiment, the extracts, fractions, and compounds of this invention may be administered in conjunction with other additives and fillers known to those of skill in the art. Other compatible actives may be included in the food compositions of the present invention.

Pharmaceutical Compositions

In an embodiment of the invention, a pharmaceutical composition for treating a disease state associated with neuroinflammation and/or oxidative stress comprises a source of ergothioneine, a UV irradiated, enriched mushroom, tissue, substrate or component thereof with higher levels of Vitamin D2 than a non-irradiated product, and a pharmaceutically-acceptable carrier. The pharmaceutical compositions according to the invention may comprise enriched mushrooms from a variety of fungi sources as disclosed according to embodiments herein this description. The pharmaceutical compositions may optionally exclude turmeric, omega-3 or an alternative antioxidant.

The pharmaceutically-acceptable carrier according to the invention facilitates administration of the composition to a patient in need thereof. The turmeric, ergothioneine and the compound, extracts, fractions and/or compounds derived therefrom the enriched mushrooms of the invention may be mixed with any of a variety of pharmaceutically-acceptable carriers for administration. “Pharmaceutically acceptable” as used herein means that the extract, fraction thereof, or compound thereof or composition is suitable for administration to a subject to achieve the treatments described herein, without unduly deleterious side effects in light of the severity of the disease and necessity of the treatment. According to the invention, the carrier may be a solid or a liquid, or both, and is preferably formulated with the compound as a unit-dose formulation, for example, a tablet, which may contain from 0.5% to 95% by weight of the active compound.

The pharmaceutical composition according to the invention may be prepared by any of the well-known techniques of pharmacy consisting essentially of admixing the components, optionally including one or more accessory ingredients. In one embodiment, the extracts, fractions, and compounds of this invention may be administered in conjunction with other medicaments known to those of skill in the art. Other compatible pharmaceutical additives and actives may be included in the pharmaceutically acceptable carrier for use in the compositions of the present invention.

Dose ranges of the pharmaceutical compositions can be adjusted as necessary for the treatment of individual patients and according to the specific condition treated. Any of a number of suitable pharmaceutical formulations may be utilized as a vehicle for the administration of the compositions of the present invention and may be a variety of administration routes are available. The particular mode selected will depend of course, upon the particular formulation selected, the severity of the disease, disorder, or condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, sublingual, transdermal or parenteral routes and the like. Accordingly, the formulations of the invention include those suitable for oral, rectal, topical, buccal, parenteral (e.g., subcutaneous, intramuscular, intradermal, inhalational or intravenous) and transdermal administration, although the most suitable route in any given case will depend on the nature and severity of the condition being treated and on the nature of the particular active product used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by admixing the compound with water or a glycine buffer and rendering the resulting solution sterile and isotonic with the blood. Alternately, the extracts, fractions thereof or compounds thereof can be added to a parenteral lipid solution.

Formulations of the inventive mixtures are particularly suitable for topical application to the skin and preferably take the form of an ointment, cream, lotion, paste, gel, spray, aerosol, or oil. Carriers which may be used include Vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin) through the skin. For this, the dosage form typically takes the form of an optionally buffered aqueous solution of the active compound. Suitable formulations comprise citrate or bis/tris buffer (pH 6) or ethanol/water and contain from 0.01 to 0.2M active ingredient.

The therapeutically effective dosage of any specific compound will vary somewhat from compound to compound, patient to patient, and will depend upon the condition of the patient and the route of delivery. As a general proposition, a dosage from about 0.01 to about 50 mg/kg will have therapeutic efficacy, with still higher dosages potentially being employed for oral and/or aerosol administration. As one skilled in the art will ascertain, dose ranges can be adjusted as necessary for the treatment of individual patients and according to the specific condition treated. Toxicity concerns at the higher level may restrict intravenous dosages to a lower level such as up to about 10 mg/kg, all weights being calculated based upon the weight or volume of the enriched mushrooms, fractions thereof or compounds thereof of the present invention, including the cases where a salt is employed.

Methods of Use

Embodiments of the invention include methods of increasing cellular longevity and/or cellular rejuvenation and tolerance to chronic inflammation and oxidative stress in animals, methods of decreasing neuroinflammation and increasing resistance to oxidative stress and associated disease states in animals, and methods of treating a disease state associated with neuroinflammation and/or oxidative stress, including neurodegenerative diseases associated with increased amyloid precursor protein or production of free radicals in animals, such as Alzheimer's Disease.

According to an embodiment of the invention, a method of increasing cellular longevity and/or cellular rejuvenation and tolerance to chronic inflammation and oxidative stress in animals comprises administering to said animal a source of turmeric, ergothioneine and a naturally enhanced, filamentous fungi, tissue, substrate, spent substrate or component thereof, with increased levels of Vitamin D, wherein upon administration of the same, cellular longevity and/or cellular rejuvenation is increased. Optionally, according to additional embodiments of the invention the source of turmeric can be omitted, however additional benefits as disclosed according to the invention are achieved through the use of turmeric.

According to a further embodiment of the invention, a method of decreasing neuroinflammation and increasing resistance to oxidative stress and associated disease states in animals comprises administering to said animal an effective amount of turmeric, ergothioneine and a filamentous fungi that has been naturally enriched in Vitamin D2.

A still further embodiment of the invention includes a method of treating a disease state associated with neuroinflammation and/or oxidative stress, including increased amyloid precursor protein or production of free radicals in animals comprising administering to said animal with said disease state a composition comprising turmeric, ergothioneine and a pulsed UV irradiated, filamentous fungi, tissue, substrate, spent substrate or component thereof, with increased levels of Vitamin D2, wherein upon administration of the same, survivability of said animal is increased when compared to an animal with such disease state without such treatment.

A further embodiment of the invention is the antioxidant and cytoprotectant efficacy achieved in animals as a result of administering to animals an effective amount of the compositions according to the invention. The methods of the invention provide antioxidant cytoprotection as a result of enhancing the protection of mitochondrial components (e.g. DNA) from oxidative damage. Still further the methods of the invention provide effective prevention, suppression and/or treatment of conditions resulting from oxidative damage of proteins, lipids and DNA. The methods of the invention are effective in preventing, suppressing and/or treating the release of ROS in order to further prevent, suppress and/or treat resultant inflammation in a variety of tissues, including the brain.

Another embodiment of the invention is the use of the compositions to provide ergothioneine to prevent DNA damage as a result of internal ROS production in the cell and/or mitochondria, an organelle within the cell. According to the invention the compositions providing ergothioneine neutralize the ROS species.

Methods of use according to the invention may include administration of the compositions, food products, supplements and/or pharmaceutical compositions on a daily basis, weekly basis, or other frequency for the particular purpose. Although not intending to be limited to a particular theory of the invention, it is believed that daily administration of the turmeric, ergothioneine and Vitamin D enriched mushrooms will benefit a variety of disease states associated with inflammation and oxidative stress. Daily supplementation is preferred for those with significant risk for disease states associated with inflammation and oxidative stress, such as traumatic brain injury or PTSD, so that they are preloaded with the bionutrients and have elevated serum, cellular, and storage levels in order to protect against acute and chronic effects of the conditions. According to this embodiment, daily supplementation reduces levels of ROS as well as the signs and symptoms of inflammation theorized to cause PTSD, depression, traumatic brain injury, and other disease states.

Applicants demonstrated that the combination of antioxidants, including phytonutrient turmeric and ergothioneine, along with Vitamin D enriched mushrooms increase cellular longevity in Drosophila kept under nutritionally deficient diet. These results represent a novel use of the compositions of the invention for treating a variety of disease states associated with inflammation and oxidative stress. According to the invention, Applicants have shown that the compositions increase survival and decrease biologic death in conditions associated with oxidative stress, which include disease states such as Alzheimer's disease and other associated diseases including those involving chronic markers of inflammation, such as chronic depression, traumatic brain injury and PTSD. Thus the supplements, food compositions and pharmaceutical compositions according to the invention, employing the Vitamin D enriched mushrooms, ergothioneine, and optionally turmeric and/or other antioxidants have surprising benefits for treatment of such disease states.

The various embodiments of the invention, including methods of use or administration of compositions for the treatment of inflammation and oxidative stress or disease states or conditions associated therewith, are useful for a variety of subjects. Mammals may be treated using the methods of the present invention and are typically human subjects. According to additional embodiments, the methods of the present invention may be useful for veterinary purposes with other animal subjects, particularly mammalian subjects including, but not limited to, horses, cows, dogs, rabbits, fowl, sheep, and the like. According to additional embodiments, an animal is any non-human primate, such as for example, a cow, horse, pig, sheep, goat, dog, cat, rodent, fish, shrimp, chicken, and the like.

Without being limited to a particular theory of the invention and the benefits provide therein, Applicants demonstrate the common underlying basis of the various conditions afforded prevention, suppression and/or treatment benefits according to the invention, including for example PTSD, TBI and other co-morbid states whose basis is an inflammatory response in brain tissue. Without limiting the scope of the invention, free radicals and/or ROS are causative agents in inducing inflammation. This is confirmed by the fact that aging, senescence and death of cells and tissues are directly associated with a rise in intracellular ROS and a loss of telomerase reverse transcriptase (TERT) activity. There has been compelling evidence regarding the direct association of ROS and TERT with inflammation and cell damage. Further description is set forth in Haendeler et al., Antioxidants Inhibit Nuclear Export of Telomerase Reverse Transcriptase and Delay Replicative Senescence of Endothelial Cells. Circ. Res., 2004; 94: 768-775. These antioxidants (e.g. N-acetylcysteine and statins) have been shown to block free radical actions of H2O2, reduce intracellular ROS formation and prevent mitochondrial damage.

There has been further illustrated the direct involvement of ergothioneine and the ergothioneine transporter with mitochondrial protection. In particular the amino acid has demonstrated physiologic cytoprotectant effects along with the transported ergothioneine being heavily concentrated in mitochondria. (Paul & Snyder, The Unusual Amino Acid L-Ergothioneine is a Physiologic Cytoprotectant. Cell Death & Differentiation, Nov. 13, 2009). Mitochondria are cytoplasmic organelles responsible for life and death. Evidence from animal and clinical studies suggest that mitochondria play a critical role in aging, cancer, diabetes and neurodegenerative diseases such as Alzheimer's disease and Parkinson's disease. The research indicates that depleting cells of the ergothioneine transporter causes cells lacking ETT to be more susceptible to oxidative stress. The oxidative stress results in increased mitochondrial DNA damage, protein oxidation and lipid peroxidation.

There is a definite causative association between free radicals, oxidative stress, mitochondrial damage and dysfunction, and neurodegenerative diseases. According to the invention, certain antioxidants, such as mushroom-based Ergothioneine and Vitamin D2 could offer a healthy intervention in prevention of diseases or palliation of diseases.

Methods of Detection

In an aspect of the invention methods of detecting or diagnosing the ergothioneine transporter provides for the identification of susceptible individuals. Susceptible individuals may include those having a genetic predisposition to lack or absence of the ergothioneine transporter. Still further, susceptible individuals may include those having specific neurological disease that are associated with different levels, decreased or increased levels in the ergothioneine transporter.

A further aspect of the invention may include the measuring or detecting of concentrations of ergothioneine and/or the ergothioneine transporter in tissues in patients. Diagnostic tests for the measurement of the presence, absence, and/or decreased concentration of ergothioneine and/or the ergothioneine transporter are further included within the scope of the invention. The diagnostic tests may optionally be included in the methods steps of the invention to determine patients in need of treatment thereof.

The method of detection and/or measurement according to the invention can be achieved through the assaying of cell tissue for the presence of the ergothioneine transporter. For example, conditions such as Crohn's disease, regional ileitis, and ulcerative colitis can be evaluated by studying tissue biopsies for presence, absence, and/or levels of the ergothioneine transporter. This approach can be used in any normal or cancerous tissue available to biopsy study, such as prostate, breast, etc. Transporter levels can also be compared in a patient to levels of inflammatory cytokines, such as chemokines, interleukins, etc. According to a further embodiment, the transporters levels of various biomarker paradigms could be evaluated to determine the presence, absence and/or levels. For example, macrophages and white blood cells from the blood of a patient can be evaluated to measure the ergothioneine and ergothioneine transporter. Additional suitable biomarkers may include but are not limited to amyloid precursor protein (APP), c-reactive protein (CRP), interleukins, complement, and markers of brain death such as caspase 3 and macrophages. A paradigm of diagnosis and need for ergothioneine supplementation can be created as a result of such assaying and comparison to inflammatory markers.

In an aspect of the invention, this diagnostic capability is suitable for use to identify the various conditions in the assayed tissue (e.g. brain) in the applicable patient. According to embodiments of the invention, these diagnostic steps are suitable for identifying conditions in need of prevention, suppression and/or treatment, such as TBI or Alzheimer's disease. The identification of the ergothioneine transporter within the tissues and the presence (or absence) of ergothioneine provide an indication of whether the compositions of the present invention are indicated. For example, a person identified as having a particular neuroinflammatory and/or oxidative stress-related condition could be preloaded with the ergothioneine using the compositions according to the invention.

Another aspect of the invention includes the isolation of macrophages and white blood cells from the blood of a patient. This aspect of the invention provides significant convenience and logistics for use of the methods of the invention, as tissue samples or biopsies are not always available (e.g. brain tissue biopsy from a living patient). As a result, the isolation of macrophages and white blood cells from the blood of a patient creates a paradigm for use of the invention. Upon isolation according to methods well known by those in the art, the degree and presence of both the ergothioneine transporter and ergothioneine can be determined to identify a condition in need of treatment using the compositions and methods of the invention.

Still further, cells that are isolated can be removed and grown in tissue cultures and thereafter reintroduced into a patient's system after the stimulation of the ergothioneine transporter production by loading ergothioneine, for example. The present invention also provides the ability to modify and/or reprogram somatic or adult stem cells in these disease states through loading with ergothioneine.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Pulses of UV radiation of approximately 1-10 J/cm² per pulse, preferably 3-8 J/cm² and most preferably 5-6 J/cm² are used to UV-enhance Vitamin D and/or its derivatives in filamentous fungi. Voltages may also vary based upon safety concerns but should generally be in the range of 1 to 10 or even up to 100 or 10,000 volts as safety mandates. The pulses should generally be in a range of 1-50 pulses per second more preferably 1-30 pulses per second and most preferably 1-10 pulses per second for a range of treatment post-harvest of 0 to 60 seconds.

The inventors used 5.61 J/cm² per pulse on the strobe surface for an input voltage of 3800V and with 3 pulses per second. Sliced mushrooms (Agaricus bisporus, white strain) were placed in the pulsed UV-light sterilization chamber and treated with pulsed light for up to a 20-second treatment at a distance of 17 cm from the UV lamp or 11.2 cm from the window. Control samples did not undergo any pulsed UV treatment. Treated mushrooms were freeze-dried and then sent to a selected commercial laboratory for Vitamin D2 analysis. In this study, a pulsed UV system was also evaluated for effects on the appearance of fresh mushroom slices during a shelf life study.

Results of the experiments demonstrated that pulsed UV-light was very effective in rapidly converting ergosterol to Vitamin D2. Control mushrooms contained 2 ppm d.w. Vitamin D2, while 10 and 20 seconds of exposure to pulsed UV-light resulted in 17 and 26 ppm Vitamin D2, respectively (FIG. 1). This increase was equivalent to over 1800% DV Vitamin D in one serving of fresh mushrooms after a 20 second exposure to pulsed UV (FIG. 2). The mushrooms treated for 20 seconds also showed no noticeable difference in appearance initially as well as after 10 days of storage at 3° C. compared to the untreated control.

These results compared favorably to the previous pilot study (Feeney, 2006) where mushrooms were exposed to 5 minutes of conventional UV-light exposure. In that study, the mushrooms contained 14 ppm Vitamin D2, but they were also significantly discolored. Hence, the pulsed UV method shows considerable promise as a rapid means to enhance Vitamin D2 levels in fresh mushrooms, theoretically reducing required exposure times from minutes to seconds. Pulsed UV-light exposure did not result in any negative effects on mushroom quality.

Another experiment revealed that pulsed UV-light could rapidly convert ergosterol present in dried oyster mushroom powder to Vitamin D2 (Table 1). These findings indicate that this technology could be used to enrich other mushroom products with Vitamin D2.

TABLE 1
Vitamin D2 generation in dried oyster mushroom powder exposed to
pulsed UV-light (C-type lamp).

| Time of Exposure(s) | Vitamin D2 (PPM) |
|---|---|
| 0 | 8.5 |
| 8 | 15.18 |
| 16 | 24.24 |

The filamentous fungi product is subjected to pulsed UV irradiation after harvest, being irradiated with UV light for a time sufficient to enhance the Vitamin D content thereof. By utilizing UV irradiation, the food product has a substantially increased level of Vitamin D. Preferably, the food product is irradiated with UV radiation, specifically Ultraviolet-B (UV-B), a section of the UV spectrum, with wavelengths between about 280 and 320 nm, or Ultraviolet-C (UV-C), with wavelengths between about 200 and 280 nm. In a more preferred embodiment the UV radiation is pulsed. It is believed that the additional Vitamin D is obtained through the conversion of ergosterol due to the UV irradiation. The time may be the same or increased when the irradiation occurs during the growing process, or post-harvest though the UV irradiation can occur during both periods.

### Example 2

The effect of Agaricus blazei (1-4) on the survival rate of Drosophila melanogaster fed a nutritionally deficient diet, at room temperature (22° C.) was tested using the following parameters: Agaricus blazei (no UV treatment): 1.6 g Vitamin D2/g, dry weight; two pulses of UV B light: 241.0 g Vitamin D2/g, dry weight; plain yeast paste base as control; vials containing 5.0 ml 1% Agarose medium; yeast paste containing 3% w/w concentration of the two samples.

Drosophila is a model organism with an experimental history of over 100 years. It has a life cycle (embryo to adult) of about 12 days at 22° C. and 9 days at 25° C. The adults live for about 85 days at 22° C. and 60 days at 25° C. under laboratory conditions. It has 3 major chromosomes. Drosophila and human development are homologous processes. They utilize closely related genes working in conserved regulatory networks. Unlike humans, Drosophila can be genetically manipulated. As a result, most of what we know about the molecular basis of animal development has come from studies of model systems such as Drosophila. Drosophila has nearly all the important genes that vertebrates including humans have. Not only the genes are conserved but the pathways regulated by these genes are also conserved. A reliable model using Drosophila as a system to evaluate the effect of a compound for survival on nutritionally deficient diet has been developed by Dr. Krishna Bhat.

The effect of Agaricus Blazei without enrichment, with Vitamin D2 enrichment, pure Vitamin D2 and control (vehicle for the delivery) on the survival rate of Drosophila melanogaster under Paraquat-induced oxidative stress condition was tested. The study focused on the control of Paraquat induced oxidative stress/biologic death. Paraquat is a very potent oxidative stress inducing chemical and causes death in animals and plants by the toxicity of released free radicals. Paraquat (10 mM concentration) (Sigma Aldrich) was used to chemically induce oxidative stress. Paraquat is the trade name for N,N′-dimethyl-4,4′-bipyridinium dichloride, a widely used herbicide. Paraquat, a viologen, is quick-acting and non-selective, killing green plant tissue on contact. It is also toxic to humans when swallowed. This is the most standard chemical used in experimental induction of oxidative stress using the Drosophila model system. It catalyzes the formation of reactive oxygen species (ROS). Paraquat will undergo redox cycling in vivo, gets reduced by an electron donor such NADPH, before being oxidized by an electron receptor such as dioxygen to produce superoxide, a major ROS.

The following materials and methods were utilized. Vials containing 10 mM Paraquat (from Sigma Aldrich) in 5 ml of 1.2% Low melting point Agarose medium were prepared. A strip of half moist filter paper was inserted in the medium (with the wet end in). Yeast paste containing 1% concentration (w/w) of the various test materials (see above) mixed to homogeneity was prepared. Yeast paste without drug was used as control. Uniform aliquot (˜300 mg) of yeast paste with or without the test material) was applied to vials in such a way that yeast paste was on the glass surface and covered the dry end (top) of the filter paper strip. Freshly enclosed wild type isogenized Canton-S males and females were collected and starved on 1% agar medium for 5-6 hours. Four males and females were transferred to the vial containing 10 mM paraquat in LMP agarose medium and yeast paste with +/− test material (8 flies per vial). 6 vials were used per experiment. Vials with flies were placed horizontally in a tray. The experiment was conducted at 25 degrees C. temperature. Flies were transferred once in 2 days and the number of flies surviving at each transfer was recorded.

Results: Over a period of 10 days, flies fed yeast paste containing A. blazei with Vitamin D2 enrichment showed marked and significant survivability under Paraquat-induced oxidative stress condition compared to the control yeast paste alone (54%+/−10% versus 23%+/−8%), yeast paste containing A. blazei without the vit D2 enrichment (54%+/−10% versus 27%+/−8%), and yeast paste containing pure Vitamin D2 (54%+/−10% versus 13%+/−3%). Vitamin D2 in its pure form had a deleterious effect on the survival and therefore seems to aggravate the oxidative stress.

The results show that a combination of naturally induced Vitamin D2 together with the components of A. blazei has the highest potential and activity to suppress the oxidative stress from Paraquat. These results are highly significant, showing that Vitamin D2, produced naturally by mushrooms, was active only when present within the parent whole food; Vitamin D2 and Vitamin D3 by themselves (i.e. single nutrient or pure Vitamin D2 and Vitamin D3) had no beneficial effect. Oxidative or inflammatory stress was dramatically induced in the Drosophila fruit fly model by the toxic agent, Paraquat, and the end-point of death was evaluated. This model is a very well established paradigm to evaluate oxidative stress. These findings show a novel use for A. blazei enriched with Vitamin D2 for suppressing oxidative stress and associated biologic death. The results are shown in FIGS. 1 and 2.

### Example 3

A. blazei enriched with Vitamin D2 significantly were analyzed to determine whether they enhance the survival and life span of Alzheimer's disease (AD) model in Drosophila. The study evaluated the ability of the edible specialty mushrooms according to the invention, with and without naturally enhanced levels of organic Vitamin D2, to extend the lifespan of the Alzheimer's disease mutant fruit fly.

Type of Model (with specific Drosophila model of neurodegeneration, with references). The targeted over/ectopic expression of APP in the brain using a UAS promoter driven APP transgene, induced by a specific GAL4 trans-driver in the brain of a Drosophila model system, was used for this Example. Below is a reference for such over-expression of APP in the Drosophila model system and the combination gives a fully penetrant AD with limited life-span.

β-Amyloid peptides and amyloid precursor protein (APP) play a deterministic role in Alzheimer's disease (AD). In Drosophila, the targeted expression of the key genes of AD, APP, causes generation of β-amyloid plaques and age-dependent neurodegeneration as well as progression to semi lethality, a shortened life span; genetic manipulations or pharmacological treatments with secretase inhibitors influenced the activity of the APP-processing proteases and modulated the severity of the phenotypes (GREEVE I., et al., 2004; The Journal of neuroscience 24, 3899-3906). The AD strain lives only for a few days after their eclosion (birth) as opposed to 65 days or more for wild type normal strains.

We determined the extension of life span in the mutant strain for each test compound. We used a specific GAL4 driver that induces the APP gene in the central brain area at high levels (see above) and results in a fully penetrant lethality within a 2-3 weeks period. When these AD flies are given A. blazei enriched with Vitamin D2, the survival rate was increased nearly double that of the control or A. blazei without any enrichment. See FIG. 3. Treating AD flies with pure Vitamin D2 or Vitamin D3 had no such effect. These results indicate that components in A. blazei, in combination with UV-enriched natural Vitamin D2 has significant benefit against the AD disease.

### Example 4

A series of experiments were run according to the methods earlier reported on Paraquat induced oxidative stress and Drosophila Alzheimer's disease flies. We used the targeted over/ectopic expression of APP in the brain using a UAS promoter driven APP transgene, induced by a specific GAL4 trans-driver in the brain of Drosophila model system. Since we used a strong GAL4 inducer to activate the hAPP, a significant lethality occurred between one to two weeks after eclosion as opposed to 65 days or more for wild type normal strains. We determined the extension of life span in the mutant strain for each test compound as detailed below.

Procedure: Freshly eclosed virgin females from UAS-hAPP strain and males from 408-GAL4 strain were collected and mated in bottles containing cornmeal agar media. Flies were allowed to lay eggs for 3-4 days at 25 degrees C. temperature. Then, the parent flies were transferred to fresh media. The bottles containing eggs from the cross were transferred to 18 degrees C. chamber and allowed to grow until eclosion. Freshly eclosed (virgin) heterozygous (F1) male and female progeny from the cross were collected separately and starved for 5-6 hours in vials containing 1% agar media.

In the meantime, yeast paste containing required concentration of compound was prepared. For mushroom powder, 1% w/w concentration was used. For Vitamin D2 and VitD3, 75 mg/10 gm yeast (or 0.75% w/w) concentration was used. Required quantity of both yeast and compound was weighed and finely ground in a pestle and mortar. The finely ground powder was transferred to a small beaker and appropriate quantity of water was added and mixed very well to make a homogeneous paste.

About 300 mg aliquot of yeast paste with/without compound was uniformly applied to the wall of the vial and touching media. Moist filter paper strip was placed inside the vial to maintain humidity. After 5-6 hours of starvation, four males and four females (UAS-hAPP;408-GAL4) were transferred to each vial containing 1% agar medium with yeast paste (plus or minus compound). These flies were transferred to fresh vials containing same 1% agar medium with yeast paste (plus or minus compound) on every alternate day. This experiment was conducted at 25 degrees C. temperature and the vials were scored for surviving/dead flies at every transfer. Graphs were plotted using the mean percentage survival on alternate day for the treated versus non-treated flies.

The results show that naturally enriched Vitamin D2 mushrooms have the ability to increase biologic survival and nutritionally prevent biologic death as compared to the same unenriched mushroom. The enriched mushrooms further resulted in increased survival when compared to Vitamin D2 and Vitamin D3 alone. Vitamin D3 actually decreased survival. A. blazei enriched mushrooms had better long term survival than Agaricus bisporus, but both had better effects that non-enriched mushrooms.

### Example 5

Experiments testing the anti-inflammatory effects of mushroom-based formulations with increased natural levels of Vitamin D2 according to the invention were tested in an equine inflammatory gum disease study. Elderly horses with inflammatory gum were treated with a mushroom-based formulation, 10 grams per day, for 30-60 days; formulations contained increased levels of Vitamin D2.

Horses showed dramatic improvement in the severity of the gum disease within 30-60 days as shown in FIG. 4. A separate 30 day clinical study, involving 36 horses, fed mushroom-based formulations, revealed a statistically significant increase in numbers of white blood cells; mean response among the study sample was 12%. This percentage increase in white blood cells within a 30 day period after dietary supplementation is further supportive evidence for improvement in the animal's immune response and ability to suppress inflammatory diseases, such as gum disease.

Inflammatory disease of the gums is a perfect example of the inflammatory process that occurs in other tissues and/or organ systems, such as arteries, nerves, heart, colon, and brain, to name a few. The terms inflammation, free radicals, reactive oxygen species (ROS) and oxidative stress are almost interchangeable and a clear understanding of the interactive processes has uncovered new approaches to prevention and amelioration of inflammation and or inflammatory disorders no matter what the origin or location. Similar to the inflammatory processes involved in gum disease, free radicals can perpetuate tissue and organ damage and the disease itself. PTSD, TBI and depression have inflammation as a major root cause of the diseases process and should be responsive to amelioration of the process with anti-inflammatory mushroom-based formulations, such as those shown to be effective in equine gum disease.

### Example 6

A patient study is performed using the following measurements to analyze the beneficial impact on daily supplementation of Vitamin D2, ergothioneine and an antioxidant (such as turmeric) on disease states associated with inflammation, free radicals and oxidative stress. The use of a novel whole food dietary supplement, named ERGO-D Traum™, containing potent antioxidants and anti-inflammatory bionutrients demonstrates beneficial clinical effects on the prevention and/or suppression of PTSD, based upon the following clinical markers. The ERGO-D Traum™ product contains the following formulation: Agaricus blazei and turmeric.

Patients undergo standard cognitive testing, including the following: PTSD checklist (military version, developed by the National Center for PTSD); State-Trait Anger Inventory; Pittsburgh Sleep Index; and OQ-45 quality of life index (measures symptom distress which are heavily weighted in anxiety and depression; interpersonal relations; and social role).

Patients further undergo digital thermal imaging, wherein IR images are collected to detect changes in heat signatures of the head/neck region associated with inflammation.

Blood testing is also collected to detect changes in biomarkers associated with inflammation and brain injury, including but not limited to amyloid precursor protein (APP), c-reactive protein (CRP), interleukins, complement, and markers of brain death such as caspase 3 and macrophages.

It anticipated that patients with PTSD, TBI, and depression will show improvement in physical and mental clinical outcomes including blood biomarkers following dietary supplementation with proprietary combinations of Agaricus blazei and Turmeric.

Separate clinical experiments will be performed in mice and rats by preloading the animals with similar proprietary Ergo-D Traum™ formulations and assessing tissue and organ response to traumatic brain injury. The aim of these studies is to show the value of preloading an animal with Ergo-D Traum™ to ameliorate and/or suppress nerve damage that occurs with direct trauma. Appropriate inflammatory biomarkers will also be measured. It is anticipated that the results will confirm our other previously performed studies and show that dietary supplementation with specific antioxidants and bionutrients, prior to or after brain injury, will result in decreased brain tissue damage.

### Example 7

Freshly eclosed virgin females from UAS-hAPP strain and males from 408-GAL4 strain were collected and mated in bottles containing cornmeal agar media. Flies were allowed to lay eggs for 3-4 days at 25° C. temperature. Then, parent flies were transferred to fresh media. The bottles containing eggs from the cross were transferred to 18° C. chamber and allowed to grow until eclosion. Freshly eclosed (virgin) heterozygous (F1) male and female progeny from the cross were collected separately and starved for 5-6 hours in vials containing 1% agar media.

In the meantime, yeast paste containing required concentration of compound was prepared. For turmeric powder, 1% w/w concentration was used. Required quantity of both yeast and compound was weighed. The finely ground powder was transferred to a small beaker and appropriate quantity of water was added and mixed very well to make a homogeneous paste.

About 300 mg aliquot of yeast paste with/without compound was uniformly applied to the wall of the vial and touching media. Moist filter paper strip was placed inside the vial to maintain humidity. After 5-6 hours of starvation, four males and four females (UAS-hAPP;408-GAL4) were transferred to each vial containing 1% agar medium with yeast paste (plus or minus compound). These flies were transferred to fresh vials containing same 1% agar medium with yeast paste (plus or minus compound) on every alternate day. This experiment was conducted at 25° C. temperature and the vials were scored for surviving/dead flies at every transfer.

Graphs were plotted using the mean percentage survival on alternate day for the treated versus non-treated flies. The results show that turmeric powder has the ability to increase survival of AD flies. The results are discussed further below and are shown in FIG. 5. Moreover, a combination of turmeric and Vitamin D2 enriched mushroom also had the ability to increase survival of AD flies, as shown in FIG. 6.

### Example 8

The expression pattern of SLC22A4 was evaluated in a variety of human tissues and diseases through immunohistochemistry. The results provide further explanation to explain the physiologic and potential health protective role of ergothioneine.

Antibodies developed to SLC22A4 were evaluated on formalin-fixed, paraffin-embedded (FFPE) positive and negative control cell lines and a multi-tissue array of human normal tissues to identify the best reagents and concentrations for use in immunohistochemistry. The cell lines tested were a positive cell line TNCS1a-ETTh, which expresses SLC22A4, and a negative control cell line TNCS1a-CTTh, which expresses carnitine transporter. The human multi-tissue block included cores of the following normal tissues: adrenal, brain, breast, colon, heart, small intestine, kidney, liver, lung, skeletal muscle, pancreas, placenta, prostate, skin, spleen, testis, thymus, thyroid, tonsil, and uterus.

Antibody titration experiments were conducted with all 5 antibodies; formalin-fixed, paraffin-embedded human tissues were supplied by LifeSpan and control cell lines (ETTh and CTTh) supplied by Entia Biosciences, Inc. (Dr. Dirk Gründemann) prepared by LifeSpan. The 5 antibodies were initially applied to the tissues and a proprietary LifeSpan detection system was then used including a Vector Red substrate kit to produce a fuchsia-colored deposit, identifying the presence of the SLC22A4 transporter. The slides were interpreted by a pathologist and each antibody was evaluated for the presence of specific signal, level of background, and concordance with expression results reported in the literature.

Staining was recorded on a 0-4 scale (0=negative, 1=blush, 2=faint, 3=moderate, 4=strong). Slides stained at the reported concentrations or dilutions were imaged with a DVC 1310C digital camera coupled to a Nikon microscope. Images were stored as TIFF files with Adobe Photoshop. The antibodies that were negative were imaged at the highest titers. Preferred antibodies to show specific, positive membranous and granular cytoplasmic staining for the Ergothioneine Transporter (ETT) were identified and are suitable for use in diagnostic methods of the invention.

Results indicate that ETT is highest in concentration in the most undifferentiated and rapidly dividing tissues. For example, undifferentiated stem cells were illustrated in the testing of a normal placenta of an 18-year old female patient. The placenta image showed significant staining of the cytotrophoblast. The cytotrophoblast is considered to be the trophoblastic stem cell; it differentiates into the other forms of trophoblastic tissue (intermediate trophoblast and syncytiotrophoblast). The intermediate trophoblast is more highly differentiated and anchors the placenta to the maternal tissue. The syncytiotrophoblast is the epithelial covering of the placenta villous tree. These cells differentiate and secrete hormones to maintain the integrity of the uterine lining.

The cells of the intestinal lining were tested as representative of cells and tissues that rapidly divide. The absorptive and protective functions of the intestines are dependent on an intact and functional epithelium. This epithelial layer undergoes continuous and rapid replacement of the differentiated cells by replication of somatic adult undifferentiated stem cells located within intestinal crypts. This process of cell renewal is based upon a limited number of long-lived multipotent intestinal stem or progenitor cells. These cells are similar to those of the hematopoietic system and also the hair follicle. These cells must have two main properties: self-renewal or the ability to maintain itself throughout long periods of time and the potential to generate all differentiated cell types present within a tissue or organ.

The results indicate that the levels of ergothioneine are higher in the mitochondria of rapidly dividing tissues. A non-limiting theory of the invention and explanation is that rapid cell division exposes the cell to free radicals and oxidative damage and the antioxidant protective role of ergothioneine is needed. In addition, ergothioneine levels are directly related to different tissue requirements for mitochondrial function. For example, placenta, intestinal linings and hematopoietic tissues have high energy requirements as compared to other tissues in the body.

### Example 9

Another key cell involved in inflammatory response within the brain is the astrocyte. As one of skill in the art understands, astrocytes are a specialized type of glial cell, and are the primary support cells of the brain and spinal cord. Astrocytes make and secrete neurotropic factors necessary for neurons to survive and they provide an extra storehouse of energy for hard-working neurons. Additionally, astrocytes break down and remove proteins or chemicals that could be harmful to neurons, like extra neurotransmitters, especially the neurotransmitter glutamate, which can cause neurons to become overexcited and die by a process called excitotoxicity. After an injury to the CNS, astrocytes divide (or proliferate) to make new cells and surround the injury site, making a barrier called a glial scar.

Neuron death by excitotoxicity corresponds to various neuro-inflammatory conditions, including for example traumatic brain injury and strokes. The immunohistochemistry results outlined in Example 8 provide evidence of the role of ergothioneine levels in tissues. Tested tissues illustrated a response of astrocytes (e.g. lighting up on stained tissue) with the ergothioneine antibody presenting a first look at this correlation between conditions such as traumatic brain injury and stroke and the role of ergothioneine in preventing or treating such excitotoxicity.

### Example 10

Further evidence of the role of astrocytes and excitotoxicity (as discussed in Example 9) is illustrated in Alzheimer's disease. The confirmation that astrocytes are activated with increased ergothioneine is significant. According to an aspect of the invention, tissues and cells have the potential to be reprogramed. For example, a cell could be modified to increase ergothioneine transporter levels to increase the ergothioneine within the cells and tissue. In one aspect, such reprogramming can be effectuating using stem cells as they have not yet differentiated into the adult somatic cell. This provides a nutrigenomic, personalized medicine approach to preventing, suppressing and/or treating various conditions associated with inflammation, ROS and/or oxidative damage.

### Example 11

A second phase of studies was conducted to focus on analyzing the presence/levels of SLC22A4 in a whole array of normal tissues and cancer tissues, including but not limited to breast, colon, lung, ovary, prostate, pancreas and skin. The studies focus on neurodegenerative diseases, such as Parkinson's disease, Alzheimer's disease, Multiple sclerosis, and Lou Gehrig's disease, Crohn's disease, ulcerative colitis, and metabolic syndrome, including diabetes. The tests provide further evidence of the benefits afforded by administration of the compositions of the invention. In particular the studies on ergothioneine and the ergothioneine transporter (SLC22A4) demonstrate the indication for compositions of the invention to regenerate and/or treat tissues and organs that have been damaged by oxidative stress and inflammation.

An antibody was selected for this study to be used as the primary antibody at a concentration of 2.0 μg/ml. Tissues were also stained with positive control antibodies. The negative control consisted of performing the entire immunohistochemistry procedure on adjacent sections in the absence of primary antibody. The slides were interpreted by a pathologist and each antibody was evaluated for the presence of specific signal and level of background. Staining was recorded on a 0-4 scale (0=negative, 1=blush, 2=faint, 3=moderate, 4=strong). Slides stained at 2.0 μg/ml were imaged with a DVC 1310C digital camera coupled to a Nikon microscope. Images were stored as TIFF files with Adobe Photoshop.

Results

Within the CNS, when comparing the staining results of diseased tissues with their corresponding normal tissue counterparts, the most significant difference in staining was observed in stroke samples. Neurons within normal cortex and within unaffected regions of patients with infarct showed relatively uniform, faint or moderate staining, and astrocytes were mostly negative or showed faint staining. Within stroke samples, there was both increased variability of staining, as well as clusters of strongly positive neurons adjacent to areas of infarction or necrosis, and adjacent to neurons that showed diminished staining. This was also often accompanied by infiltrations of moderately to strongly positive macrophages and reactive astrocytes in areas of infarct.

Increased staining in occasional neurons was also rarely observed in Alzheimer's samples, although in these cases, this was accompanied by slightly more variability of staining within neurons, so it is difficult to determine if neurons showed increased staining, or neighboring neurons were decreased relative to those that remained moderately positive.

Alzheimer's cases showed occasional faintly positive plaques and one case showed faint staining of amyloid in affected vessels with amyloid angiopathy. The majority of senile plaques were negative for staining in the three Alzheimer's cases. Slightly increased staining was also observed in astrocytes in these cases.

Multiple sclerosis cases showed increased numbers of positively staining macrophages and increased staining within reactive astrocytes. No significant differences in staining were observed in dopaminergic neurons Parkinson's disease substantia nigra, but increased numbers of positively staining macrophages were observed in these cases.

Amyotrophic lateral sclerosis spinal cords showed no significant differences in staining compared to normal spinal cord. Within inflammatory bowel disease cases, increased numbers of positively staining plasma cells and macrophages were observed in areas of ulceration and inflammation, and slight increases in staining within reactive capillaries. In inflammatory diseases where increased numbers of positively staining macrophages and plasma cells were observed, there was both an increase in the level of positive staining within the average inflammatory cell, as well as many more positively staining inflammatory cells. The increased staining within macrophages was most evident at the base of ulcers, where large collections of macrophages and cellular debris were often strongly positive for staining.

Results—Brian, Cortex, Normal

Sample 1: This sample of cerebral cortex was obtained from a 50-year-old patient of unknown sex who died of Hodgkin's lymphoma. Sections showed normal cortex from a 50 year old who died of Hodgkin's lymphoma. Neurons within all layers of the cortex showed faint to moderate granular cytoplasmic staining, with slightly greater staining of pyramidal and larger neurons. Faint staining was also observed in capillaries, with faint staining of occasional endothelial cells and pericytes. Faint to moderate staining was observed in perivascular macrophages. In muscular vessels of the meninges, endothelium was variably negative to faint to occasionally moderate, and vascular smooth muscle was moderately positive. Within glia, the majority of astrocytes and oligodendroglia within the white matter were negative or showed rare faint granular staining, and occasional microglia were faintly positive. Subpial astroglia were moderate.

Sample 2: This sample of cerebral cortex was obtained from an 84-year-old female with hypertension who died of chronic obstructive pulmonary disease. Sections showed normal cortex. Neurons within all layers of the cortex showed faint to moderate granular cytoplasmic staining, with slightly greater staining of pyramidal and larger neurons. Faint staining was also observed in capillaries, with faint staining of occasional endothelial cells and pericytes. Faint to moderate staining was observed in perivascular macrophages. Within glia, the majority of astrocytes and oligodendroglia within the white matter were negative. Occasional astrocytes or microglia showed rare faint granular staining

Results—Brain, Hippocampus, Normal

Sample 1: This sample of hippocampus was obtained from a 52-year-old female who died of cardiopulmonary decompensation. Sections showed normal hippocampus. Antibody showed moderate to strong positive granular staining of neurons within regions CA 2-4, faint staining of CA-1 and blush to faint staining of dentate gyrus neurons, and occasional faint staining of neurons within the subiculum and adjacent gray matter. Staining of neurons within the hippocampus diminished from the regions of CA-1 to adjacent gray matter of the subiculum and parahippocampal cortex. Faint staining was also observed in capillaries, with faint staining of occasional endothelial cells and pericytes. Faint to moderate staining was observed in perivascular macrophages. In muscular vessels of the meninges, endothelium was faint to occasionally moderate, and vascular smooth muscle was faintly to moderately positive. Within glia, the majority of astrocytes and oligodendroglia within the white matter were negative, or showed rare punctate nuclear staining, and occasional microglia were faintly positive. Meningothelial cells showed faint to moderate staining, and the choroid plexus was faint to moderate. Ependymal cells were negative or showed blush staining

Sample 2: This sample of hippocampus was obtained from a 37-year-old female who died of an uncharacterized, poorly differentiated malignant neoplasm. Sections showed normal hippocampus. Antibody showed moderate to strong positive granular staining of neurons within regions CA 1-4, faint staining of dentate gyrus neurons, and diminishing staining between CA 1 and the subiculum, with occasional faint staining of neurons within subiculum and adjacent parahippocampal gyrus. Faint staining was also observed in capillaries, with faint staining of occasional endothelial cells and pericytes. Faint to moderate staining was observed in perivascular macrophages. In muscular vessels of the meninges, endothelium was faint to occasionally moderate, and vascular smooth muscle was faintly to moderately positive. Within glia, the majority of astrocytes and oligodendroglia within the white matter were negative or showed rare faint granular nuclear staining, and occasional microglia were faintly positive. Ependymal cells were negative or showed blush staining

Results—Brain, Substantia Nigra, Normal

Sample 1: This sample of normal substantia nigra was obtained at autopsy from an 86-year-old female. The section showed normal substantia nigra from an 86 year old female. Pigmented neurons were moderately positive for staining Non-pigmented neurons were also moderately positive. Astrocytes and macrophages showed occasional moderate staining. The majority of oligodendrocytes were negative. Within vessels, endothelial cells and pericytes showed occasional faint granular staining.

Sample 2: This sample of normal substantia nigra was obtained from a 52-year-old male with alcoholic end-stage liver disease who died of hepatic failure. The section showed normal substantia nigra from a 52 year old male. Pigmented neurons were moderately positive for staining Non-pigmented neurons were also moderately positive. Occasional astrocytes and macrophages showed moderate staining. The majority of oligodendrocytes were negative. Within vessels, endothelial cells and pericytes showed occasional faint granular staining

Brain, Alzheimer's Disease (Cortex and Hippocampus)

Sample 1: This sample of brain was obtained at autopsy from a 72-year-old female as depicted in FIGS. 7A-C. Sections showed hippocampus from a patient with Alzheimer's disease. Antibody showed faint to moderate to strong positive granular perinuclear, cytoplasmic staining of neurons within regions CA 1-4, faint staining of dentate gyms neurons, and faint staining with occasional moderate staining of neurons within the subiculum and adjacent parahippocampal gyrus. Senile plaques, showed faint to occasional moderate staining of plaque material. Neurons showing granulovacuolar degeneration were faintly positive. Within the white matter, subsets of astrocytes and microglia showed moderate staining Faint staining was also observed in capillaries, with faint staining of occasional endothelial cells and pericytes. Faint to moderate staining was observed in perivascular macrophages. Subpial astroglia were moderate. In muscular vessels of the meninges, endothelium was negative to faint, and vascular smooth muscle was faintly to moderately positive. Compared to normal brain samples, this sample of Alzheimer's disease showed faintly positive senile plaques, and slightly increased numbers of positive astrocytes.

Sample 2: This sample of brain was obtained at autopsy from an 88-year-old female as depicted in FIGS. 8A-C. Sections showed cortex from a patient with Alzheimer's disease. Antibody showed moderate to strong positive granular staining of subsets of pyramidal and occasionally smaller neurons. Rare senile plaques showed occasional faint to moderate staining of plaque material. Vessels showing amyloid angiopathy showed occasional faint staining of amyloid. Faint staining was also observed in capillaries, with faint staining of occasional endothelial cells and pericytes. Faint to moderate staining was observed in perivascular macrophages and astrocytes. In muscular vessels of the meninges, endothelium was faint to occasionally moderate, and vascular smooth muscle was faintly positive. Compared to normal brain samples, this sample of Alzheimer's disease showed faint staining of vessels with amyloid angiopathy, rare faintly positive senile plaques, and increased numbers of positive astrocytes.

Sample 3: This sample of brain was obtained at autopsy from a 78-year-old male as depicted in FIGS. 9A-C. Sections showed cortex from a patient with Alzheimer's disease. Antibody showed moderate to strong positive granular staining of subsets of pyramidal and occasionally smaller neurons. Rare senile plaques showed occasional faint staining of plaque material. Most plaques were negative for staining Faint staining was also observed in capillaries, with faint staining of occasional endothelial cells and pericytes. Moderate staining was observed in perivascular macrophages and astrocytes. Compared to normal brain samples, this sample of Alzheimer's disease showed variable staining of neurons, and increased numbers of positive astrocytes.

Results—Brain, Stroke (Cortex)

Sample 1: This sample of brain was obtained at autopsy from an 85-year-old female. The sample showed a section of cortex with an infarct encompassing most of the section. Residual surviving neurons showed variable staining ranging from moderate to strong, with moderate staining of astrocytes, and moderate staining of neuropil. Areas of infarction were heavily infiltrated with moderately to strongly positive macrophages, and moderately positive reactive astrocytes. Reactive capillary endothelial cells were also moderately positive for staining Vessels showed moderate staining of endothelium and vascular smooth muscle. Areas of necrosis showed strongly positive macrophages surrounded by moderately staining cellular debris. Compared to normal brain, this section showed increased staining within neurons, macrophages, reactive astrocytes, and capillaries in areas of infarction.

Sample 2: This sample of brain was obtained at autopsy from an 81-year-old female. The sample showed a section of cortex with an infarct encompassing most of the section. Residual surviving neurons showed variable staining ranging from faint to moderate. Areas of infarction showed patchy collections of strongly positive neurons, accompanied by moderately positive reactive astrocytes. Collections of macrophages in areas adjacent to necrosis were moderately positive. Occasional oligodendroglia also showed faint staining Vessels showed faint to moderate staining of endothelium and vascular smooth muscle. Compared to normal brain, this section showed increased staining within neurons adjacent to areas of necrosis, and increased staining within macrophages and reactive astrocytes.

Sample 3: This sample of brain was obtained at autopsy from a 67-year-old male. The sample showed a section of cortex with an infarct encompassing most of the section. In areas away from the infarct, neurons ranged from faint to occasionally moderate. In areas adjacent to the infarct, residual surviving neurons showed moderate to strong staining. Areas of infarction showed moderate staining of macrophages and moderately positive reactive astrocytes. Occasional oligodendroglia also showed faint staining. Microglia were also moderate. Vessels showed faint to moderate staining of endothelium and vascular smooth muscle. Compared to normal brain, this section showed increased staining within neurons adjacent to areas of infarction, and increased staining within macrophages and reactive astrocytes.

What is claimed is:

1. A method of increasing cellular longevity, cellular rejuvenation and/or tolerance to chronic inflammation and oxidative stress in animals comprising:
administering to said animal a source of ergothioneine and a naturally enhanced, filamentous fungi, tissue, substrate, spent substrate or component thereof, with increased levels of Vitamin D, wherein upon administration of the same, cellular longevity, cellular rejuvenation and/or tolerance to chronic inflammation and oxidative stress is increased.

2. The method of claim 1 further comprising detecting an ergothioneine transporter in tissue sample of said animal to confirm a need for said source of ergothioneine to increase cellular longevity, cellular rejuvenation and/or tolerance to chronic inflammation and oxidative stress.

3. The method of claim 1 wherein said Vitamin D is Vitamin D₂.

4. The method of claim 1 wherein said filmentous fungi is a mushroom.

5. The method of claim 4 wherein said mushroom is of a species selected from the group consisting of: Agaricus bisporus, Agaricus blazei, Lentinula edodes, Pleurotus ostreatus and Pleurotus eryngyi, and wherein said mushroom is enriched by pulsed UV irradiation without changing said mushroom's ergothioneine content.

6. The method of claim 4 wherein said fungi is in powder form.

7. The method of claim 1 further comprising administering a source of turmeric and/or antioxidant, wherein said turmeric is in powder form.

8. The method of claim 3 wherein said Vitamin D₂ content is increased to about 800% of the daily recommended value of Vitamin D.

9. A method of decreasing neuroinflammation and increasing resistance to oxidative stress and associated disease states in animals comprising:
administering to said animal an effective amount of ergothioneine and a filamentous fungi that has been naturally enriched in Vitamin D₂.

10. The method of claim 9 further comprising detecting an ergothioneine transporter in tissue sample of said animal to confirm a need for said source of ergothioneine to decrease neuroinflammation and increase resistance to oxidative stress and associated disease states.

11. The method of claim 9 wherein said enrichment is from UV treatment.

12. The method of claim 9 wherein said filamentous fungi is a mushroom selected from the group of species consisting of: Agaricus bisporus, Agaricus blazei, Lentinula edodes, Pleurotus ostreatus and Pleurotus eryngyi.

13. The method of claim 9 wherein said mushroom is selenium enriched.

14. The method of claim 9 further comprising administering a source of turmeric and/or antioxidant, wherein said turmeric and fungi are in powder form.

15. The method of claim 9 wherein said Vitamin D₂ content is increased to about 800% of the daily recommended value of said vitamin.

16. A method of treating a disease state associated with neuroinflammation and/or oxidative stress, including increased amyloid precursor protein or production of free radicals in animals comprising:
administering to said animal with said disease state a composition comprising ergothioneine and a pulsed UV irradiated, filamentous fungi, tissue, substrate, spent substrate or component thereof, with increased levels of Vitamin D2;
wherein upon administration of the same, survivability of said animal is increased when compared to an animal with such disease state without such treatment; and
wherein said filmentous fungi is a mushroom selected from the group of species consisting of: Agaricus bisporus, Agaricus blazei, Lentinula edodes, Pleurotus ostreatus, and Pleurotus eryngyi and said mushroom is selenium enriched.

17. The method of claim 16 further comprising detecting an ergothioneine transporter in tissue sample of said animal to confirm a need for said source of ergothioneine to treat a disease state associated with neuroinflammation and/or oxidative stress.

18. A nutritional product for increasing cellular longevity, cellular rejuvenation and/or tolerance to chronic inflammation and oxidative stress in animals comprising:
ergothioneine; and
a UV irradiated, filamentous fungi, tissue, substrate or component thereof with higher levels of Vitamin D than a non-irradiated product.

19. The nutritional product of claim 18 further comprising omega-3 and/or turmeric.

20. A pharmaceutical composition for treating a disease state associated with neuroinflammation and/or oxidative stress comprising:
a source of ergothioneine;
a UV irradiated, Agaricus fungi, tissue, substrate or component thereof with higher levels of Vitamin D2 than a non-irradiated product; and
a pharmaceutically-acceptable carrier.

21. The pharmaceutical composition of claim 20 wherein said product comprises Agaricus blazei, wherein said Agaricus blazei comprises higher levels of Vitamin D2 than a non-irradiated product and is in a powder form.

22. The pharmaceutical composition of claim 20 further comprising omega-3 and/or turmeric.
